(19) United States
(12) Patent Application Publication
(10) Pub. No.: US 2003/0166715 A1
(43) Pub. Date: Sep. 4, 2003

(54) Use of vitaletheine modulators in the prophylaxis and treatment of disease

(76) Inventors: Galen D. Knight, Albuquerque, NM (US); Terence J. Scallen, Albuquerque, NM (US)

Correspondence Address: JEFFREY D. HSI, Fish & Richardson P.C., 225 Franklin Street, Boston, MA, 02110-2804, US

(21) Appl. No.: 10/006,798

(22) Filed: Dec. 4, 2001

Related U.S. Application Data
(63) Continuation of application No. 08/466,143, filed on Jun. 6, 1995, now abandoned.
Application No. 08/466,143 is a continuation of application No. 07/910,892, filed on Jul. 10, 1992, now abandoned.
Application No. 07/910,892 is a continuation-in-part of application No. 07/549,105, filed on Jul. 6, 1990, now abandoned.

(51) Int. Cl. A61K 31/27; A61K 31/17; A61K 31/155

(52) U.S. Cl. 514/478; 514/588; 514/634

(57) ABSTRACT

Sulfur-containing derivatives of carboxy-amino-amides are provided for use, inter alia, in treating diseases or disorders.

### BACKGROUND OF THE INVENTION

1. Field of the Invention

The invention relates to the use of novel short-chain carboxylic acid containing thiol amides, and their corresponding disulfides, as well as compounds representing intermediate and other oxidation states of these thiols such as sulfenic acids and sulfenyl halides; tautomers, oligomers, and rearrangement forms of these compounds; and further derivatives or forms as described hereinafter. The seminal compound is vitaletheine,

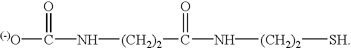

The compounds, referred to herein as “vitaletheine modulators”, are useful in the clinical treatment of a number of superficially disparate diseases or disorders caused by underlying disruption of particular metabolic pathways, especially those dependent upon the regulation of thiol- or disulfide-dependent enzymes, both intracellular and membrane-bound.

The modulators have demonstrated efficacy in such superficially diverse applications as adaptation of cells such as natural killer (NK) cells to culture; promotion of erythropoiesis in vitro; and the treatment of neoplasia. The compounds have contemplated efficacy, inter alia, in the treatment of AIDS, lupus erythematosus, rheumatoid arthritis, atherosclerosis, hypercholesteremia, diabetes, cystinosis, and progeria, and to maintain or enhance erythropoiesis.

2. Discussion of Related Art

It is well-known that endogenous thiols and disulfides are critical to the function of a multitude of dependent enzyme systems in the body (1,2), including thiol-dependent and disulfide-dependent branch-point enzymes controlling access to major metabolic pathways. Glutathione (GSH, gamma-glutamyl-cysteinylglycine, an acid tripeptide thiol) is the most abundant thiol in mammalian cells, and an entire regulatory and regenerating system ensures an adequate supply of this reducing agent (3,4,5), which maintains and buffers cell thiol/disulfide ratios. Coenzyme A (CoA) and lipoic acid are prevalent in mammalian systems and also regulate dependent enzyme activity. Xenobiotic thiols such as dithiothreitol (DTT, Cleland's reagent) or dithioerythritol are routinely used experimentally to regulate activity of thiol-dependent enzymes.

In response to demand, thiols such as GSH, CoA, and lipoic acid can, for example, activate thiol-activatable enzyme by reducing inactive oxidized (disulfide) enzyme to the corresponding thiol with a concomitant oxidation of the activating thiol to its corresponding disulfide (GSSG in the case of glutathione-GSH) according to the following scheme, wherein P is protein:

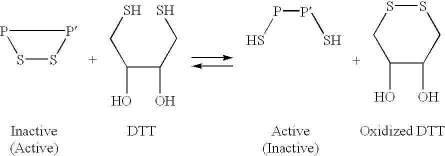

Activity of thiol-dependent enzymes is a function of the availability of the thiols involved as expressed by the thiol/disulfide ratios of their thiol/disulfide redox buffers (upper arrow); interaction is complex, however, and activity is further dependent on additional factors such as substrate, ambient ions, and type of reducing thiol (membrane-bound enzymes, for example, are resistant to reduction by glutathione). Similarly, activity of disulfide-dependent enzymes (in parenthesis) is a function of the availability of disulfides, as expressed by the thiol/disulfide ratios of their redox pairs (lower arrow).

By the above mechanisms, endogenous thiol/disulfide redox buffers such as GSH/GSSG systems control the activity of many critical enzymes; thyroxine monodeiodinase is exemplary of thiol-dependent enzymes. Regulation of the activity of this enzyme by thiol/disulfide buffer controls the induction of a host of important enzymes, including HMG-CoA reductase, the branch-point enzyme for the isoprenoid pathway, which in turn regulates the production of essential isoprenoids such as steroid hormones, dolichol, cholesterol, and ubiquinone and the isoprenylation of proteins (6,7,8,9,10,11). Glycolysis is also controlled by thiol-dependent and disulfide-dependent enzyme systems; phosphofructokinase, for example, is inactivated by disulfides, whereas fructose-1,6-bis-phosphatase with the reverse enzyme activity is activated by certain disulfides. Thiol-dependent enzymes also directly and/or indirectly control isoprenoid and oligosaccharide biosynthesis and the synthesis and utilization of thyroid hormones.

One mechanism which has been identified as participating in the in vivo regulation of thiol/disulfide equilibria is the oxidation of thiol to disulfide catalyzed by microsomal flavin-containing mixed function monooxygenase (herein referred to as “monooxygenase”). This monooxygenase catalyzes, for example, the oxidation of cysteamine to its corresponding disulfide, cystamine. Monooxygenase activity thus appears to be critical to the regulation of at least some thiol- and disulfide-dependent enzymes in vivo.

Carboxylic-acid containing thiols having an atomic spacing between the carboxylic acid moiety and the thiol moiety of from 7 to 5, (6 carbons and 1 nitrogen or 4 carbons and 1 nitrogen in the case of glutathione) have been shown to competitively inhibit the monooxygenase, and glutathione for example, is generally believed to control the availability of thiols and disulfides for downstream dependent enzymes at least to some extent by this mechanism.

Thiols, particularly glutathione and cysteine or N-acetyl-cysteine, have been demonstrated in vivo to inhibit neoplasia (Am. J. Med 91(3C):122S-130S, 1991); to inhibit replication of HIV in cell cultures (Proc. Natl. Acad. Sci. USA 87(12):4884-8, 1990); to be markedly elevated in preneoplastic/neoplastic hepatocytes (Mol. Carcin. 2(3):144-9, 1989); to influence the proliferation of human peripheral blood lymphocytes (HPBL) and T-cells (Am. J. Med 91(3C):140S-144S, 1991) to reverse inhibition of lymphocyte DNA synthesis by glutamate in cells from HIV-infected patients (Int. Immunol. 1(4):367-72, 1989); to reduce infectivity of herpes virus in vitro (Acta. Virol. Praha. 11(6): 559-61, 1967); to suppress HIV expression in monocytes (Proc. Natl. Acad. Sci. 88:986-990, 1991); and to be systemically deficient in HIV-infected individuals (Biol. Chem. Hoppe Seyler 370:101-08, 1989 and The Laucet ii:1294-97, 1989). Regulation of HMG-CoA reductase activity by thiols and disulfides is well-known; as noted above, thyroxine monodeiodinase is a thiol-dependent enzyme, and this enzyme controls the induction of HMG-CoA reductase (Eur. J. Biochem. 4:273-278, 1968). Hypercholesteremia and atherosclerosis, leading factors in heart disease, are now clearly linked to HMG-CoA reductase activity, and treatment of these conditions with various regulators of HMG-CoA reductase is known. HMG-CoA reductase activity is also linked to neoplasia, most recently by evidence of its role in the transformation of cells by activation of Ras protein which regulates oncogene expression (Adv. Enzymol. 38:373-412, 1973; Biochem. Soc. Trans. 17:875-876, 1989; Science 245:379-385, 1989; 3-Hydroxy-3-Methylglutaryl Coenzyme A Reductase, Sabine ed. CRC Press, Inc., Boca Raton, Fla., USA, pp. 245-257, 1983).

Another carboxylic-acid containing thiol peptide, pEEDCK (Blood 77(6): 1313-19, 1991), has activities similar to vitaletheine. The reduced form, pEEDCK, prevents proliferation of hematopoietic stem cells from the bone marrow, while the oxidized form (disulfide) stimulates proliferation of these cells. Thus, the disulfide can, for example, be used to induce production of normal immunocytes (Exp. Hematol. 16: 274-280, 1988). Like the disulfide of pEEDCK, the vitaletheine tetramer (V₄), contains four free carboxylic acid groups and both stimulate colony formation from progenitor cells; conversely, vitalethine and pEEDCK have two free carboxyl groups, and both suppress colony formation from progenitor cells. Vitalethine is, however, considerably more potent than pEEDCK, especially in vivo.

Vitaletheine also has a close structural relationship to tetramisole and carbalethiazolidine, vitaletheine has the same nucleophilic spacing as the carbalethiazolidines, and is one carbon longer in the spacing of its nucleophiles than tetramisole (optical isomers: levamisole and dextramisole). Vitaletheine, however, is not sterically hindered as is tetramisole and the carbalethiazolidines, so in addition to being structurally similar to these prior art compounds, vitaletheine has far greater flexibility for adaption to sterically selective receptors.

Both tetramisole and the carbalethiazolidines have demonstrated utility in the treatment of a host of diseases or disorders, including cancer, autoimmune diseases such as lupus nephritis or erythematosus, rheumatic diseases such as rheumatoid arthritis, immune deficiency diseases, parasitic diseases such as leishmania, hypersensitivity, allergy, hypertension; the compounds have utility also as antihistaminics, analgesics, anti-inflammatory agents, anti-ulcer agents, and as psychotropic, seratonergic, dopaminergic, anti-hallucinogenic, anti-schizophrenic, and anti-dysthymic agents, inter alia, in neurological disorders (see, e.g., Oncology 38:168-181, 1981; JP 82-67,585; JP 80-142870; EP 49,902; JP 80-142,869; JP 58-110,595; JP 59-88,491; FR 2,574,408; EP 144,101; FR 2,530,636; EP 49,402; JP 80-142,869; JP 57-130,988; EP 348,746; EP 45,251; JP 59-088,491; JP 63-225,383).

Additional structurally related compounds having known biological efficacy include calchemicins and esperamicins; the specie active in anticancer activity is a carboxy-amino derivative more closely related to vitaletheine than the original drug.

Hexamethylene-bis-acetamide (HMBA) and sulfide- and sulfone-containing isothiocyanates are further exemplary of drugs structurally similar to vitaletheine having biological activity, particularly anticarcinogenic activity.

Structural relationships between these compounds and vitaletheine modulators is illustrated in FIG. 4.

### BRIEF DESCRIPTION OF THE DRAWINGS

FIGS. 1A and B illustrate response of Cloudman S-91 melanoma to treatment with vitalethine modulator in a murine model;

FIGS. 2A and B are similar, except the melanoma is NS-1;

FIGS. 3A and B illustrate the effect of vitaletheine modulator on erythropoiesis; and

FIG. 4 illustrates structural relationships of vitalethine modulator with various known compounds.

### SUMMARY OF THE INVENTION

The invention provides therapies employing a group of compounds collectively referred to herein as “vitaletheine modulators” for the treatment of disease, comprising: vitaletheine, a free acid or salt of N-(2-mercapto-ethane)-[3-(carboxy-amino)-propanamide]; vitalethine, the oxidized (or disulfide) form of this compound; biologically-active or -activatable rearrangement forms of these compounds and biologically-compatible salts, hydrates, and oligomers thereof. The modulators further include biologically-active or -activatable homologs or analogs of vitaletheine or vitalethine and their corresponding rearrangement forms, including salts, hydrates, and oligomers thereof. The compounds are useful, inter alia, for promoting phenotypic expression and vitality of cells in vivo and in vitro; including, for example, the promotion of increased cellular lifespan, the promotion of increased cellular bioproductivity, and the promotion of improved cellular function.

### DETAILED DESCRIPTION OF THE INVENTION

1. The Compounds:

The compounds comprise biologically-active or -activatable sulfur-containing hydrocarbon derivatives of a carboxy-amino-amide of the Formula I, hereinafter referred to as “vitaletheine modulators” or “modulators”:

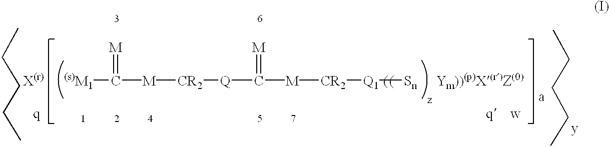

wherein:

the set of double parentheses brackets the portion of the molecule bearing a charge p when z is 1;

the expression M₁—(C═M)—M— (wherein C is the #2C) represents M₁—(C═M)—M—, M₁═(C—MA)—M—, or M₁—(C—MA)═N—, and —(C═M)—M— (wherein C is the #5C) represents —(C═M)—M— or —(C—MA)═N—; wherein A is X, −1, or a direct bond, or when —(C═M)—M— is —(C—MA)═N— or the compound is polymeric or internal cyclic or spirocyclic, A is optionally R; and M and M₁ are as defined below:

each R is independently H or a hydrocarbon radical as further defined herein;

X is a biologically-compatible cation or cationic complex as further defined herein;

X′ is a biologically-compatible ion or ionic complex as further defined herein;

M is S, O, N, or NH;

M₁ is S or O with the proviso that M₁ is also optionally N or NH when the compound is polymeric or internal cyclic or spirocyclic;

Q is CR₂ or a direct bond;

Q₁ is CR₂, CR₂CR₂, or a direct bond;

Y is O, —[C═O]—R, or a direct bond;

Z⁽⁰⁾ is a neutral moiety associated with the remainder of the compound of Formula I;

a is the absolute value of |r/(r′+pΣs)| with the proviso that when (r′+pΣs) is ≧0 , at least one q or q′ is zero such that the sum of any charges on the remainder of the complex is balanced by the charges on the ion or ions, X or X′, or the ions, X and X′;

m is 0 or a whole integer from +1 to +5;

n is 1 or 2 when z is 1, and n is 1 or 1.5 when z is 2;

p is +1, 0, or −1;

q and q′ are each independently +1 or zero;

r and r′ are each independently a whole integer from +1 to +4, or r′ is a whole integer from −1 to −4;

w is 0 or a whole integer from 1 to 5;

s is −1 or 0;

y is 1 to 40;

z is +1 or +2; and

the compound of Formula I has a molecular weight of no more than about 10,000 daltons.

Particularly interesting compounds of the Formula I are those wherein y is from 1 to about 20, especially from about 2 to 10; or wherein the number average molecular weight of the compound is no more than about 5,000 daltons, or both; and especially wherein the molecular weight of the compound is at least about 130 daltons.

Preferred compounds according to Formula I are compounds of the Formula II, herein referred to as “vitaletheine compounds”:

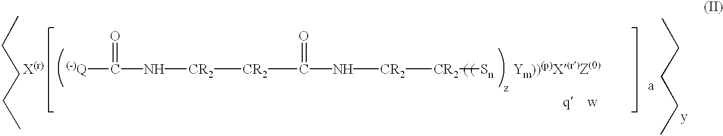

The vitaletheine compounds include compounds of the Formula II in disulfide forms, comprising homologous or heterologous (mixed) disulfides; trisulfide forms, comprising homologous or heterologous trisulfides; and oxidized forms (m>0) of the homologous or heterologous disulfides or trisulfides, wherein z is 2 and n is 1 or 1.5 according to Formula IIa:

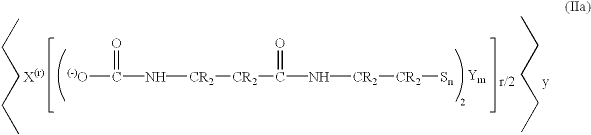

wherein R, X, Y, n, m, r and y are as defined in Formula I.

The vitaletheine compounds further include compounds of the Formula II in reduced and oxidized forms wherein z=1, according to Formula IIb:

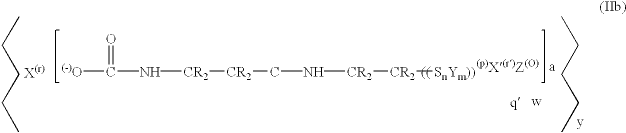

wherein R, X, X′, Y, Z, a, n, m, p, q′, r, r′, w, and y are as defined in Formula II.

Particularly contemplated radicals ((SₙYₘ))^{(p)} comprise thioesters and ionized residues of sulfoxy or S-thiosulfoxy acids, especially sulfenic, sulfinic, or sulfonic acids; and when n=2, ionized residues of thiosulfenic, thiosulfoxylic, thiosulfurous, or thiosulfuric acids. Exemplary radicals ((SₙYₘ))^{(p)} include —SOX′ (sulfenate), —SX′ (thiolate), —SI (sulfenyl iodide), —SI₃ (sulfenyl periodide), S₂O₃X′ (thiosulfate); especially SH (thiol or sulfhydryl) and SOH (sulfenic acid). As exemplified above for sulfenyl periodide, a molecule such as I₂ or H₂O, or other neutral moiety may be associated with ((SₙYₘ))^{(p)}X′(r′) or the entire monomer as Z⁰. such as I₂ or H₂O, or other neutral moiety may be associated with —((—SₙYₘ))^{(p)}(X′(r′) or the entire monomer as Z⁰.

The modulators include biologically-active or -activatable salts, hydrates, chelates, tautomers, oligomers, and rearrangement forms of the compounds of formulas I, IIa, and IIb, and the corresponding salts, hydrates and chelates of these rearrangement forms. The rearrangement forms of the compounds are primarily internal 5- or 6-membered cyclization products resulting from nucleophilic attack on susceptible atoms including oxidized sulfur and doubly-bonded carbon atoms arising from the tautomerism of the compounds as illustrated in the following Formula IIc:

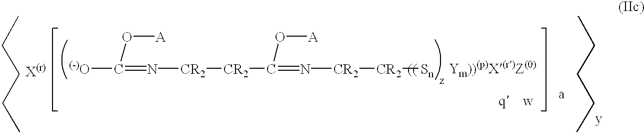

wherein R, X, X′, Y, Z, a, n, m, p, q′, r, r′ w, y, and z are as defined in Formula II; A is R, −1, a direct bond, or X; and either or both of the doubly bonded carbon atoms (2,5) are in the illustrated tautomeric form.

Compounds of the formulas I or II, wherein one or more of the atoms O, M, N, or S are rendered nucleophilic, are readily produced in vivo and in vitro where they tend to form internal cyclization products, typically stabilized by hydrogen bonds (including hydrates), ions (salts or chelates), or both. These cyclic compounds include apparently biologically-inactive but -activatable “storage” forms of compounds of the formula I or II, which are easily rearranged to the corresponding active compound. Compounds of the Formulas I and II and subformulas thereof are typically internally cyclized through S or Y, wherein p is zero, or through M₁—(C═M)—M— or —(C═M)—M—, as illustrated in the Formulas Ia′ and Ib′ and following formulas:

[image]

wherein, in the Formulas Ia′ and Ib′, M, M₁, Q, Q₁, R, X, X′, Y, a, n, m, p, q, q′, r, r′, s, w, and z are as defined in Formula I; and “c” denotes cyclization.

In general, to form a cyclic urethane of a compound of the Formula I, the charge(s) on the left terminal nucleophile M₁ (1) moves to the other nucleophile M (3), either of which may attack the doubly-bonded carbon (5) in the middle of the molecule. The developing charge on the central nucleophile M (6) then picks up an R or X group to form a urethane, or goes on to attack an oxidized sulfur atom, thereby forming a spirocyclic urethane by displacing S as illustrated in Formula Ia′; or by displacing S or Y, and X′ or Z or both X′ and Z as illustrated in Formula Ib′; in all cases z or n or both are 1 after cyclization of the compound. In a similar fashion, the central doubly-bonded carbon (5) can be attacked by one of the nucleophilic atoms S or Y (Formula 1b′), to produce a thiazolidine, or a sulfoxy or thiosulfoxy acid ester, respectively. In this latter case, a spirocyclic urethane is produced when the resulting charge on the central nucleophile (6) attacks the left terminal doubly-bonded carbon atom (2) resulting in the displacement of, for example, H₂O, H₂S, or NH₃ from the structure. Similarly, the charge or developing charge on either a central or terminal nucleophile (atoms 3 or 6, respectively) permits attack upon another monomer of the Formula I to form a dimer, which in turn is capable of polymerization to an oligomer, as described below.

Compounds of the Formula II, including the subformulas thereof, are referred to herein as “vitaletheine compounds”. The reference compound, herein referred to as “vitaletheine”, and its oxidized form, herein referred to as “vitalethine”, are believed to be the primary biologically-active forms of these compounds. Oligomers of vitaletheine containing from about 2 to about 20 monomers, preferably from about 2 to about 10 monomers, and especially from about 2 to 4 monomers are of particular interest, particularly for their stability.

Vitalethine is characterized by the structural Formula IId:

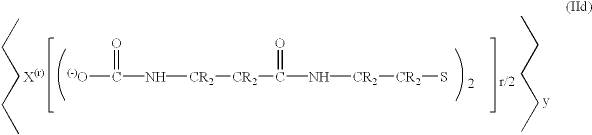

wherein R, X, r, and y are as defined in Formula II. Particularly interesting compounds of the Formula IId are those wherein R is H, and X is Zn⁺², Ca⁺², (CaI)⁺, (CaOH)⁺, or other cationic complex. The cationic groups and the hydrogen bonding illustrated in the following Formula IId′ for vitalethine (wherein y=1) appear to add overall structural stability to the otherwise labile carboxy-amino bond:

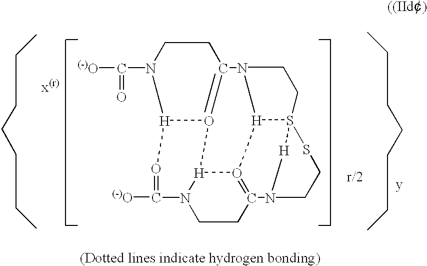

Disulfides, sulfenic acids, and sulfenates of Formula I are readily reduced to the corresponding free thiols, particularly in reactions catalyzed by endogenous enzymes, especially reductases and thiol-disulfide isomerases; in particular, vitalethine (Formula IId′) is readily reduced to vitaletheine (Formula IIe wherein R is H and y is 1):

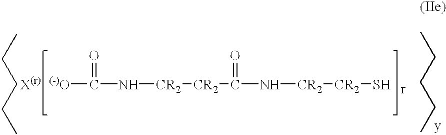

wherein R, X, r, and y in Formulas IId, IId′, and IIe are as defined in Formula II. Exemplary preferred cations X include Zn⁺², Ca⁺², or a cationic complex such as (CaI)⁺ or (CaOH)⁺, especially Zn⁺². Particularly interesting compounds include oligomers wherein y is from 2 to about 10, especially from 2 to 4, and, more especially, also wherein R is H. Oligomers of the compound of the Formula IIe wherein y is 4 appear to have great biological potency; such oligomers are referred to herein as vitaletheine V₄, which refers to compounds of the Formula IIe wherein y is 4, and more particularly refers to compounds of the Formula IIe wherein y is 4, R is H, and X is a calcium or zinc cation, or a cationic complex, as discussed in more detail below.

Exemplary biologically-activatable forms of compounds of the Formula II, which may be activatable in vivo or in vitro or converted to vitaletheine of the Formula IId or IIe, include:

1) a disulfide of a cyclic urethane of Formula IIf:

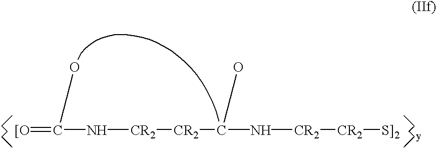

This compound appears to be stabilized as a chelate according to the following model:

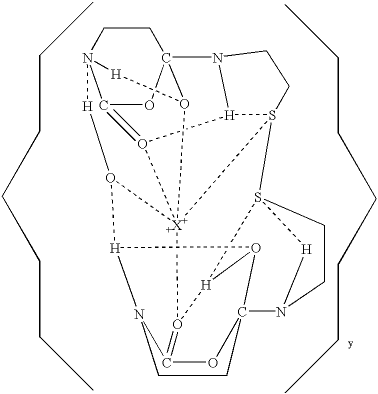

wherein R, X, and y are as defined in Formula II, especially wherein X is Mg⁺² and wherein the chelate is an Mg(OH)₂ chelate;

2) a dehydrate of compound IIf, comprising a cyclic urethane imine of the Formula IIf′:

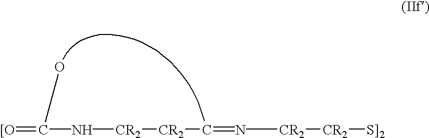

wherein R is as defined in Formula II;

3) a hydroxythiazolidine of the Formula IIg:

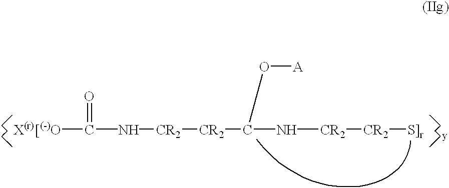

wherein X, R, y, and r are as defined in Formula II and A is R, X, a direct bond, or −1 as defined in Formula IIc;

4) a thiazoline of the Formula IIg′, in which Formula IIg is dehydrated to the thiazoline in a manner similar to the dehydration of compounds of the Formula IIf to compounds of the Formula IIf′:

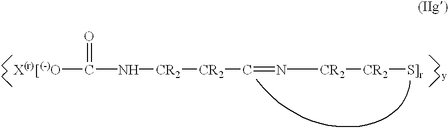

wherein X, R, r, and y are as defined in Formula II;

5) an ionized hydroxythiazolidine of the Formula IIh, as follows:

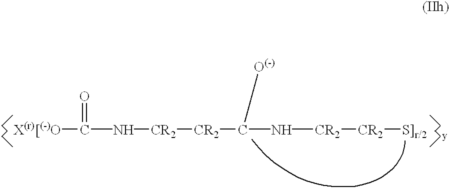

wherein R, X, r, and y are as defined in Formula II; or forms of the thiazolidine of Formula IIh in which the cyclization propagates through the carboxy-amino moiety as in Ia′ to form:

a) intermediates of the Formula IIh′:

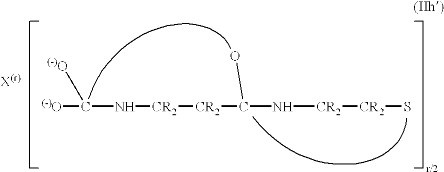

which are dehydratable to:

b) a spirocyclic urethane-thiazolidine of the Formula IIi:

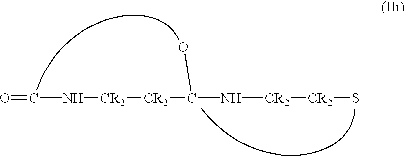

or

c) an imidocarbonate tautomer of the Formula IIi′:

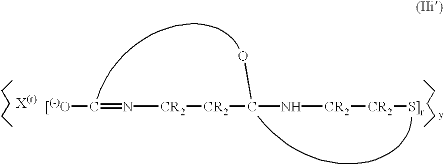

wherein X, R, r, and y in the Formulas IIh′, IIi, and IIi′ are as defined in Formula II.

Other potentially activatable rearrangement forms of vitaletheine include the following:

6) sulfenates corresponding to the cyclic urethanes of the Formulas IIf and IIf′ of the Formulas IIj and IIj′:

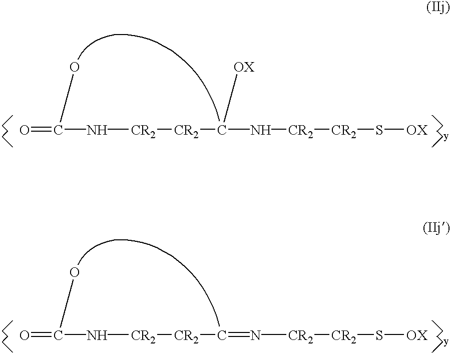

7) cyclic sulfenates corresponding to the thiazolidines of Formulas IIg, IIh, and IIh′ of the Formulas IIk, IIm, and IIm′:

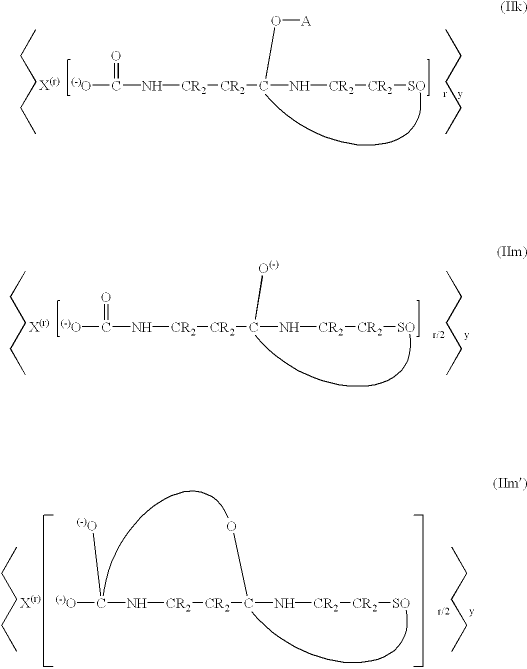

which are dehydratable to:

8) the corresponding dihydro-oxathiazine of Formula IIk′:

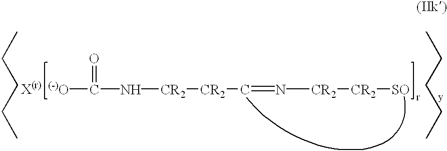

or

9) the corresponding:

a) spirocyclic urethane-sulfenate of the Formula IIn:

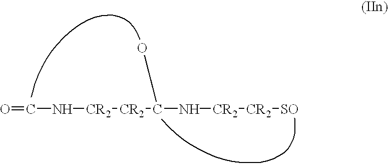

b) or the corresponding imidocarbonate tautomer of Formula IIn′:

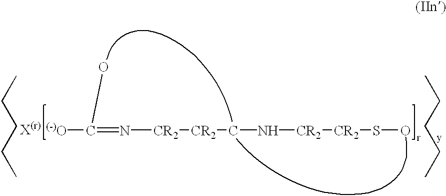

wherein X, R, r, and y in the Formulas IIj through IIn′ are as defined in Formula II, and A is as defined in Formula IIc; and the various Formulas II further include rearrangement forms as described herein, particularly as described for Formulas Ia′ and Ib′.

The modulators further comprise biologically-active and -activatable derivatives of the vitaletheine modulators of the Formula I, characterized by the following Formula III, herein referred to as “vitaletheine derivatives”:

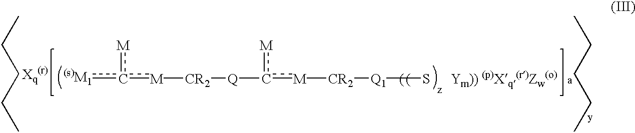

wherein M₁ is S or O; M is S, O, N, or NH; at least one M₁ or M is other than O; and R, Q, Q₁, X, X′, Y, Z, a, n, m, p, q, q′, r, r′, s, w, y, and z, are as defined in Formula I; wherein the dotted lines are bond resonances or tautomerisms; and wherein in compounds of the Formula III which are internal cyclic and spirocyclic compounds, M₁ is additionally optionally M as depicted in Formulas IV through VIe′.

Particular derivatives within the scope of Formula III include homologous or mixed sulfides, homologous or mixed trisulfides, and oxidized forms (m>0) of the homologous or mixed disulfides or trisulfides, wherein z=2 and n is 1 or 1.5 according to Formula IIIa:

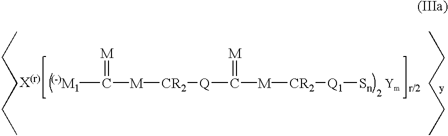

wherein M, M₁, Q, Q₁, R, X, Y, m, n, r, and y are as defined in Formula III; and X is especially H⁺, Zn⁺², calcium cation, or a calcium cationic complex.

Further derivatives within the scope of Formula III include the reduced and oxidized forms of compounds of Formula III wherein z=1, according to the Formula IIIb:

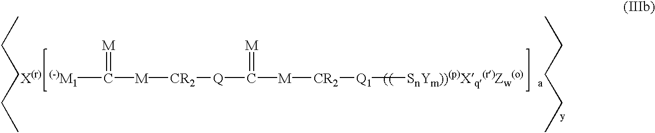

wherein M, M₁, Q, Q₁, R, X, X′, Y, Z, a, m, n, p, q′, r, r′, w, and y are as defined in Formula III, and X is especially H⁺, Zn⁺², calcium cation, or a calcium cationic complex.

The compounds of the Formula III also include these compounds in the form of their biologically-active or -activatable tautomers, chelates, hydrates, and biologically-compatible salts as described for Formulas I and II, and rearrangement products thereof, including compounds based on nucleophilic cyclization according to Formulas Ia′ and Ib′; and further include tautomeric derivatives of compounds of the Formula III as described for Formula IIc, as summarized in Formula IIIc:

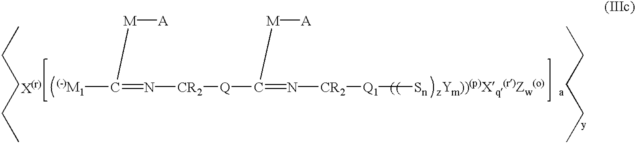

wherein M, M₁, Q, Q₁, R, X, X′, Y, Z, a, m, n, p, q′, r, r′, w, y, and z are as defined in Formula III, A is as defined in Formula IIc, and either or both doubly bonded carbon atoms (2,5) are in the illustrated tautomeric form.

Additional compounds include modulators of the Formulas IV-VI, and the subformulas thereof, wherein M₁ in the compounds of the Formula I is M:

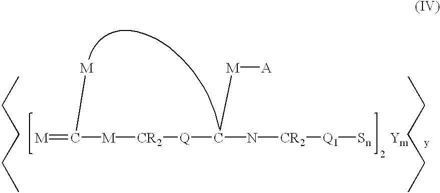

wherein M, Q, Q₁, R, Y, m, n, and y are as defined in Formula I and A is as defined in Formula IIc.

Further compounds comprise biologically-active and activatable compounds of the Formula V:

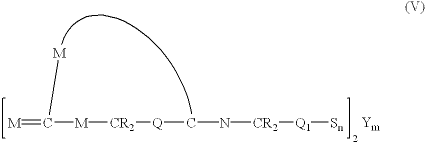

wherein M, Q, Q₁, R, Y, m and n are as defined in Formula I.

The compounds further include biologically-active and -activatable forms of compounds of the Formulas VI and the following thereof in reduced and oxidized forms, which comprise:

1) cyclic urethanes of the Formula VI:

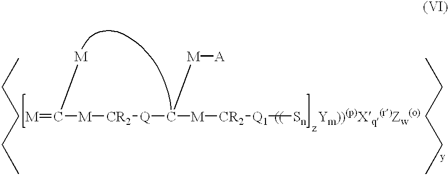

wherein the urethanes are substituted as defined in Formulas IIf, IIg, and IIh; M, Q, Q₁, R, X, X′, Y, Z, m, n, p, q′, r′, w, y, and z are as defined in Formula I, and A is as defined in Formula IV;

2) cyclic imines of the Formula VIa comprising urethanes dehydrated as analogously illustrated in Formulas IIf and IIf′:

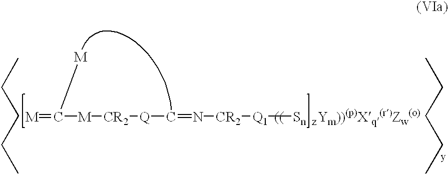

wherein M, Q, Q₁, R, X′, Y, Z, n, m, p, q′, r′, w, y, and z are as defined in Formula I;

3) spirocyclic compounds of the Formulas VIb and VIc analogous to precursors of the spirocyclic urethanes of the Formulas IIh′ and IIn:

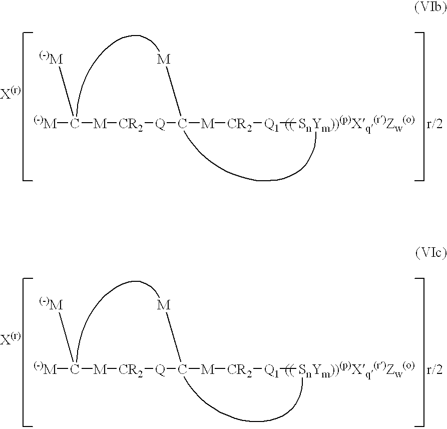

wherein M, Q, Q₁, R, X, X′, Y, Z, n, m, p, q′, r, r′, w, and z are as defined in Formula I;

4) corresponding spirocyclic urethane-sulfoxy (n=1) or urethane-thiosulfoxy (n=2) acid esters (Formula VId), or urethane-sulfides (Formula VIe), respectively, formed by elimination of sulfide, nitride, or oxide from the compounds of the Formulas VIb and VIc as H₂S, H₃N, or H₂O:

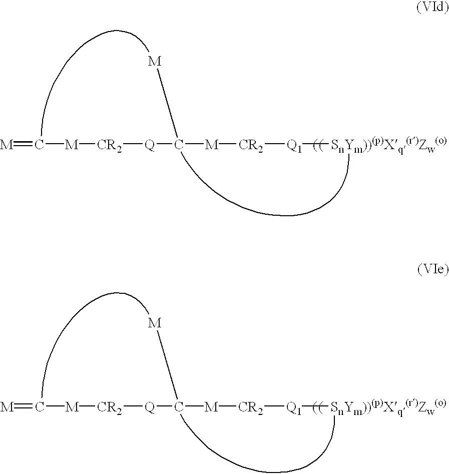

wherein M, Q, Q₁, R, X′, Y, Z, m, n, p, q′, r′, and w are defined as in Formula I; or

5) imidocarbonate tautomers of compounds of the Formulas VId or VIe, as described for Formula IIi′:

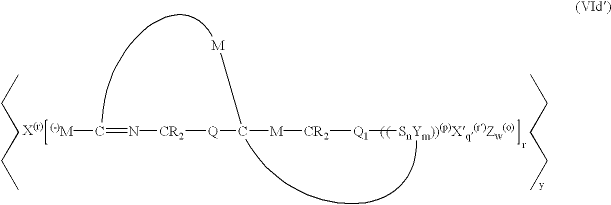

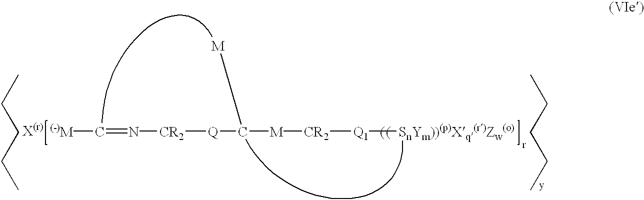

wherein M, Q, Q₁, R, X, X′, Y, Z, n, m, p, q′, r′, w, y, and z are as defined in Formula I.

The modulators especially include biologically-active or -activatable salts, hydrates, chelates, tautomers, and rearrangement forms of oligomers of monomers of the Formula I, particularly oligomers of monomers of the Formula IId, herein referred to as “vitaletheine oligomers”, comprising polymerization products of monomers of the Formula I and subformulas thereof, including cyclizations according to Formulas Ia′ and Ib′, and the corresponding salts, hydrates, tautomers, and chelates of these forms. Oligomers produced by the polymerization exemplified in Formulas Ia′ and Ib′ appear to be resistant to rearrangement and provide storage forms of compounds, which, however, may still be labile to certain organic solvents such as ethers and alcohols. Preferred oligomers of monomers of the Formula I and subformulas thereof are those wherein y is from about 2 to 10. Particularly useful preparations of vitaletheine, include those prepared, for example, according to Example III, especially those comprising a vitaletheine oligomer of 4 monomers (y=4 in Formula IIe and Formula IX following), and particularly optionally including minor proportions of at least one other oligomer or compound. This tetramer and vitalethine appear to be particularly active. Formation of this oligomer (herein referred to as “V₄”) appears to occur through an initial nucleophilic attack of a first monomer on one of the doubly-bonded carbons (2,5) of a second monomer to generate a nucleophilic oxygen from the carbonyl oxygen (6) of the second monomer. Polymerization of the monomers of Formula I and the subformulas thereof, for example oligomers wherein y is about 20 or less, appears to be propagated through this initial alkoxide ion (the nucleophilic oxygen 6 resulting from the initial dimerization) until the polymer folds back on itself and the last alkoxide ion present (the fourth in the case of V₄) reacts with the first (initiating) monomer. An intermediate dimer, exemplified in Formula VII, is comparable to a benzyl derivative of Formula VIII, obtained as a by-product under certain conditions (see, e.g., Example IIA) in the synthesis of vitaletheine V₄:

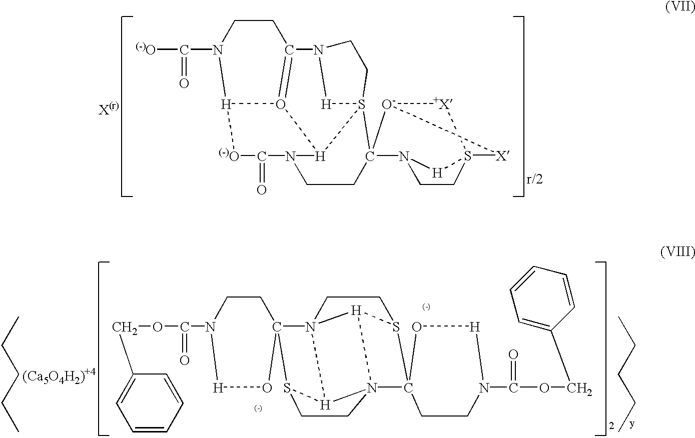

The monomers alternately are linked by Y when Y is the initial attacking nucleophile, according to Formulas Ia′, Ib′ and X.

The reaction terminating the polymerization is apparently a nucleophilic substitution of the original nucleophile involved in the formation of the first alkoxide ion by the last alkoxide ion, resulting in a cyclic polymer of monomeric subunits, which are nearly identical in spectroscopic analyses. Once formed, the polymer appears to stabilize the carboxy-amino moieties through salt bridges within the oligomer, and sterically prevents rearrangement to other active or activatable forms. Vitaletheine V₄ (the tetramer of vitaletheine, Formula IIe) is illustrated in the following Formula IX:

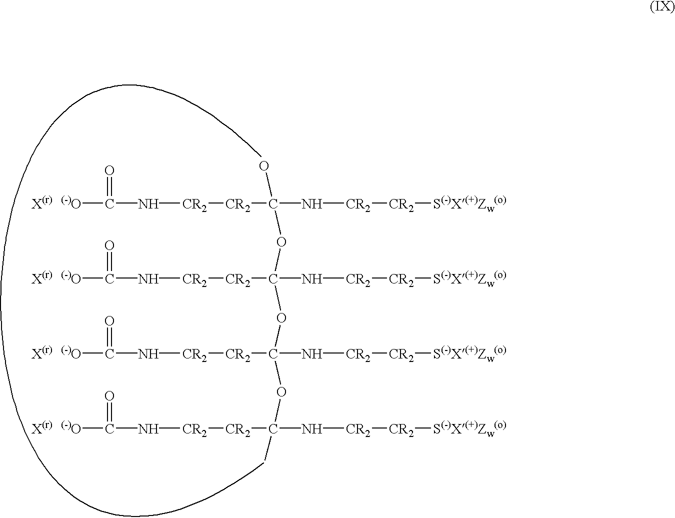

wherein R, X, X′, Z, r, [symbol] previously defined in Formula I; preferably X [symbol] of the cation Zn⁺² having a charge of +1 a pectively, is H+; and especially when X′ is a [symbol] is H⁺, r is +1, Z is H₂O, and w is 2. In the [symbol] vitaletheine V₄ as described in Example III, [symbol] neutralize the amino-carboxylate and thiolate [symbol] entire complex contains 8 moles of hydration

[symbol]

Decomposition [symbol] of vitaletheine V₄ is induced by some organic solvents such as ether, and by heating, which apparently results in decarboxylation of the polymer. Accordingly, caution should be exercised during purification procedures to obviate loss of product.

The modulators further include biologically-active and -activatable derivatives of the vitaletheine oligomers of the following Formula X, wherein a compound of Formula III is polymerized as a monomer via nucleophilic attack on one of the doubly-bonded carbons (2,5):

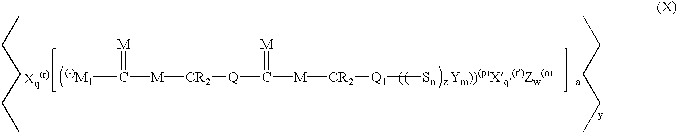

wherein the attacking nucleophile(s) comprise(s) M₁ (1), M (3,6), S, or Y as described for Formulas Ia′, Ib′, VII, VIII, and IX, and arise through the tautomerizations described herein, particularly as described for Formula IIIc; and wherein M, M₁, Q, Q₁, R, Y, X, X′, Y, Z, r, n, z, m, p, q, q′, r′, a, w, and y are as defined in Formula I.

In compounds of the Formulas I through X, and the various subformulas thereof, the hydrocarbon radical R is substituted or unsubstituted, saturated or unsaturated, with the provisos that compounds have a molecular weight of no more than about 10,000 daltons and contain less than about 40 monomers (y<40); preferably, compounds have a molecular weight of no more than about 5,000 daltons and contain less than about 20 monomers (y<20); most preferably, compounds have a molecular weight of at least about 130 daltons; compounds containing from about 2 to 10 monomers are especially interesting. Further, any hydrocarbon substituents R present must not substantially adversely affect the biofunction of the molecule, either chemically or stereochemically.

Preferably, hydrocarbon substituents R comprise suitable lipophilic moieties which counterbalance the hydrophilic portions of the molecule to promote the transfer of the modulators across the cell membrane to maximize intracellular reactions as understood by those skilled in the art. Further, R is most preferably selected to avoid stereochemical obstruction or biochemical inactivation of the active functional groups of the molecule, particularly the carboxyl-terminus and sulfur-terminus moieties which are apparently critical to the biological function of the molecule, both in their chemical constituents and their physical presentation to the cell. The substituents R are minimally function as described, do not substantially interfere with the biological activity of the molecule, do not substantially promote decomposition or unwanted side reactions of the molecule, either intracellularly or extracellularly, and do not substantially render the molecule toxic to the cell; such hydrocarbon radicals R are referred to herein as “physiologically-acceptable hydrocarbon radicals R”.

Exemplary hydrocarbon substituents R are C₁-C₂₀-hydrocarbons, especially C₁-C₁₈-aliphatic or -cycloaliphatic radicals, which are branched or unbranched, substituted or unsubstituted, saturated or unsaturated, particularly C₁-C₁₈-alkyl or -alkenyl; or substituted or unsubstituted mononuclear or polynuclear aryl, especially phenyl. An exhaustive list of potentially suitable hydrocarbon radicals R is set forth in U.S. Pat. No. 4,216,160 to Doru, et al., incorporated herein by reference, especially the hydrocarbon radicals R₁ and R₂ described therein. A particularly suitable substituent R is H.

In the compounds of the Formulas I through X, X or X′ is H⁺, hydronium, or a cation or an organic or inorganic cationic complex; or X′ is additionally an anion or an organic or inorganic anionic complex; and each X or X′ is selected for biological compatibility. The cation or cationic complex X is monovalent, divalent, or polyvalent, especially monovalent, divalent, or trivalent wherein r is +1, +2, or +3; the ion or ionic complex X′ is monovalent, divalent, or polyvalent, especially monovalent, divalent, or trivalent wherein r′ is −3 to −1 or +1 to +3. X or X′ each comprises an ion or ionic complex which does not substantially irreversibly inactivate the active portion of the molecule and which does not substantially interfere with the biofunction of the active remainder of the molecule, either chemically or stereochemically; such ions or ionic complexes X or X′ are referred to herein as “biologically-compatible ions”. Some ions may inactivate the molecule while they are present, but the inactivation is readily reversed, for example spontaneously, enzymatically, or chemically; such ions or ionic complexes are included, as it may be convenient to prepare an inactive molecule and subsequently activate it for use, especially in the preparing of molecules targeted for activation and use in specific cells or tissues. Modulators in solution are highly sensitive to electrolyte concentrations, and are easily irreversibly inactivated by excess amounts of many electrolytes, particularly magnesium ions. Further, the ions X and X′ may shift an existing equilibrium between a biologically-active form of the modulator and a corresponding storage form of the modulator in favor of the storage form, or vice versa. Exemplary cations X which appear to stabilize the molecule in either active or activatable form include Ca⁺², (CaI)⁺, (CaOH)⁺, and especially Zn⁺², which favor the active form, and Mg⁺², which may favor an activatable or storage form. Exemplary ions X′ include H⁺, I⁻, periodide (I₃⁻), Zn⁺², or Ca⁺². As described herein, a charge >+1 on the ion X or X′ may be apportioned between two or more negative charges s or p on the remainder of the molecule to form one or more salt bridges within the molecule or between molecules; the “ion X′” in this instance accordingly comprises a portion of the ion X, or vice versa. A positive ion X or X′ having a charge greater than +1 may form a bridge between a group bearing a charge of s wherein s is −1 and a group bearing a charge p wherein p is −1 in a given molecule, or between two groups bearing the charge s wherein s is −1, including molecules wherein y=1; or in molecules wherein y>1, they may form a bridge between two groups bearing a negative charge s, or two groups bearing a negative charge p, or between two groups one bearing a negative charge s and the other bearing a negative charge p. When p is +1, an ion X′ having a charge less than −1 may also form a bridge between two groups bearing a positive charge in the same molecule. Additionally, an ion X or X′ may chelate two identical or different monomers or oligomers of the Formula I. Generally, the total charges on the ions X and X′ present will balance the total charges s and p on the molecule; however, in some instances, a portion of the total charge on the molecule may be balanced by one or more ions extraneous to the molecule.

In compounds of the Formulas I through IX, the neutral moiety Zw⁽⁰⁾ is a neutral molecule or another neutral moiety which is associable with the compound of the Formula I and subformulas thereof as indicated. Exemplary neutral moieties Z_{w}⁽⁰⁾ include for example, iodine, H₂O, polyethylene glycols, and polyoxyethylene ether detergents.

Several inactive but activatable forms of the modulators within the scope of Formula I have been identified, including those described above, which appear in some instances to be inactive “storage” forms of the modulators, capable of in vivo or in vitro rearrangement to one or more active forms. in vivo rearrangement or in vitro rearrangement in the presence of living cells appears to be a result of the action of endogenous enzymes as mentioned above, which, depending upon the type of cell or cells and culture conditions, may convert inactive forms of the compounds to the corresponding active form, especially in the case of the vitalethine or vitaletheine compounds. Proteins and hydrophobic environments such as cell membranes may associate with and stabilize the active form of the product. Rearrangement of inactive but activatable forms may also be induced by other means as described below.

Within the present context, “biologically-active or -activatable” refers to compounds within the scope of Formulas I through X and the subformulas thereof which are biologically active, or which are activatable to biologically active compounds on exposure to activators such as the following: chemicals including biochemicals such as enzymes and selected organic solvents, acids, and bases; radiation including electromagnetic, actinic, or radioactive energy; or heat energy. Inactive compounds which respond to such treatment to become bioactive are referred to herein as “activatable” and are included within the scope of Formulas I through X.

Certain compounds, and other substances which are postulated to inhibit the degradation or metabolism of the modulators, are useful in combination with the modulators of Formulas I through X. At low concentrations especially, degradation catalyzed by endogenous enzymes represents a mechanism for significant losses of added modulator. Compounds which inhibit these enzymes, without themselves interfering with the action of the modulators, such as β-alethine or β-aletheine, potentiate the action of the modulator by making sustained, low, effective concentrations possible.

II. Preparation of the Compounds:

The compounds, particularly compounds of the Formula IIe wherein R is H, are postulated as endogenous to a substantially complete spectrum of plants, animals, and microorganisms, and, accordingly, it is contemplated that the compounds are recoverable from a variety of organisms and isolatable for use according to methods well-understood in the art. It is further contemplated that the recited bioapplicability of the compounds to the function of the broad spectrum of cells recited below is attributable to the ubiquitous, or near-ubiquitous presence of these compounds in virtually every living cell and the essential presence of these compounds for the autoregulation of cellular life. However, since the endogenous compounds are thought to be present, in vivo, in extremely small amounts, and are known to be easily converted into inactivatable forms, for example by customary purification methods, it is recommended that the compounds be synthesized for use, especially to avoid contamination of the product with mitogens, saponins, pathogens, antigens or other potentially reactive compounds present in biological materials, and to prevent the undesirable rearrangements described above.

At present, the most potent of these compounds appear to be those within the scope of Formula IId, viz., those based on the bis anionic [N,N′-(dithiodi-2,1-ethanediyl)-bis-(3-carboxyamino-propan-amide)] (Formula IIe) and polymers of vitaletheine. Analysis of the polymers by filtration through a P-2 gel column indicates that the monomer of vitaletheine (Formula IIe) tends to spontaneously polymerize during purification to form multimers, especially oligomers wherein y is from 2 to 4; the preparations of the V₄ oligomer and vitalethine, especially, have extremely high biological activities.

The [¹³C]-NMR of vitaletheine V₄ (Formula IIe or IX, wherein y is 4 and R is H) indicates nearly homologous subunits; the tetramer (y=4) is an extremely rigid structure similar to those reported for certain ortho-ester-like compounds in Tetrahedron Letters 22:4365-4368 [1981] (incorporated herein by reference). Based on [¹³C]-NMR analysis, the multimeric vitaletheine structures are postulated to be polymers which are formed by the attack of nucleophilic oxygen (6) derived from the central amide on the carbonyl carbon (5) of another monomer, probably through initial attack on the carbonyl carbon (5) of the amide of the initiating monomer to generate a nucleophilic oxygen (alkoxide ion) from the carbonyl oxygen (6). Polymerization may be propagated through alkoxide ions (which resemble ortho-esters), until the polymer folds back on itself and a terminal alkoxide ion reacts with the original monomer. The polymerization is then terminated by nucleophilic substitution of the atom which initiated the polymerization with a terminal alkoxide ion, resulting in a cyclic polymer which typically contains homologous monomer subunits. Slight puckering of the polymerized (—C—O—)ₙ ring (n is from about 3 to about 24, usually 3 or 4, especially 4) split observed resonances in the above-described NMR analysis of V₄ into four minor peaks in the range calculated for a highly constrained quaternary carbon atom. Polymerization of the monomer does not appear to result from manipulation of the monomer by the applied analytical procedures, since this NMR evidence indicating a tetramer was obtained prior to determination of the molecular weight of the polymer by gel filtration.

Best Modes for Preparing Compounds

Although vitalethine is also prepared by the above procedure, carboxylation of β-alethine by reacting the disulfide with phosgene in the appropriate chemical milieu is the preferred method of synthesis. Similarities in the physical properties of these two potent biomodulators, i.e. thermal lability and infrared spectra, are described in Examples III, IV and V.

The compounds were conveniently prepared employing β-alethine blocked with a protective group such as N,N′-bis-carbobenzoxy-(CBZ-) as starting material. The blocked β-alethine was then selectively deblocked to remove benzyl groups and yield the compounds. Techniques for the synthesis of the blocked β-alethine starting material are present in the literature; however, the known techniques generally provided a product of low yield or purity, or both. Many of the impurities obtained in known procedures result from the combined poor solubility of the product compound and the dicyclohexylurea by-product produced in coupling reactions which utilize dicyclohexylcarbodiimide.

Product purity and yield are improved by first coupling CBZ- or similarly-blocked β-alanine to N-hydroxysuccinimide (commercially available from Aldrich Chemicals, Milwaukee, Wis., USA) to produce the corresponding N-hydroxysuccinimide active ester using dicyclohexylcarbodiimide (commercially available from Schwarz/Mann, Orangeburg, N.Y., USA) following the procedure described in J. Am. Chem. Soc. 86: 1839-1842 (1964), incorporated herein by reference. Commercially available starting materials, such as N-CBZ-β-alanine (Sigma Chemical, St. Louis, Mo., USA), are first coupled to N-hydroxysuccinimide (Aldrich Chemicals), with precipitation or the dicyclohexylurea by-product. The soluble active ester product is recrystallized and coupled to the free amino groups of cystamine, readily obtained from cysteamine (available from Aldrich Chemicals) by oxidation with peroxide, for example, by titration in acetonitrile with peroxide until no reducing equivalents are evident. This is conveniently monitored using strips of paper soaked in a solution of 0.1M potassium phosphate buffer and 10 mM 5,5′-dithiobis-2-nitrobenzoic acid (Sigma Chemical) and dried; residual thiol in the peroxide/cysteamine mixture produces an intense yellow spot on the paper. Water added with the peroxide and produced as a by-product of cysteamine oxidation is readily removed by repeated evaporation of the acetonitrile azeotrope prior to coupling with the soluble N-hydroxysuccinimide active ester obtained by dicyclohexylcarbodiimide coupling (supra). Using this form of cystamine instead of a hydrochloride or similar salt ensures more complete reaction of the active ester with the cystamine, since this reaction is dependent upon a nucleophilic attack of the free amines of cystamine on the carbonyl carbon of the active ester. N-hydroxysuccinimide is regenerated as a by-product of this reaction as the blocked B-alethine precipitates. The benzyl groups are then removed from the blocked β-alethine as described, for example, in Examples III and IV, and the product compounds recovered.

III. Utility of the Compounds:

The vitaletheine modulators are useful, inter alia, for improving cellular phenotypic expression and cellular vitality.

Diseases or disorders projected to respond to therapies with compounds are in three general categories: 1) those diseases arising from either inadequate or excessive cell production, 2) those diseases arising from either inadequate or excessive cell function, and 3) those diseases resulting from either impaired or aberrant immunological screening. Immunological disorders such as autoimmune and immunodeficiency diseases (including hypogammaglobulinemia, candidiasis, acquired immune deficiency syndrome, lupus, and rheumatoid arthritis), aging (including progeria), thyroid-related disorders (including thyroiditis and hypo- and hyper-thyroidism), cystinosis, diabetes, and atherosclerosis and related heart disease all fall within these categories. Similarly, parasitic- and pathogen-induced disorders are included in the third category, at least until the cellular deficits or excesses of products or functions enabling these organisms to escape immunological detection and elimination are determined. The above categories also include hormonal deficiency or excess, immunological deficiency or excess, infection (including parasitic, bacterial, viral, or fungal), and premature aging. The modulators as a group are efficacious, therefore, for optimizing cellular response to disease or disorder, including hormonal imbalance, immunological challenge (such as infection or infestation), and premature senescence of cells.

Specifically contemplated cell types for modulation, according to the invention, to ameliorate disease or disorder include: cells derived from mammalian tissues, organs and glands such as the brain, heart, lung, stomach, intestines, thyroid, adrenal, thymus, parathyroid, testes, liver, kidney, bladder, spleen, pancreas, gall bladder, ovaries, uterus, prostate, and skin; reproductive cells (sperm and ova); lymph nodes, bone, cartilage, and interstitial cells; blood cells including immunocytes, cytophages such as macrophages, lymphocytes, leukocytes, erythrocytes, and platelets.

If treatment involves extraction of cells from the body, the following in vitro manipulations of extracted cells are exploitable utilities of the modulators: a) adapting to culture cells which under conventional conditions are substantially resistant to culture, i.e., those cells which have a half-life under conventional culture conditions of less than about two weeks, or which do not express normal products or normal amounts of products in culture; b) obviating the need to fuse cells to immortalizing cells capable of long-term culture in order to obtain extended bioproduction of cell products, such as the current necessity for fusing antibody-producing splenocytes or lymphocytes to immortalizing cells for the en masse production of monoclonal antibodies; c) delaying senescence of cells and the therapeutic benefits derived therefrom, in vivo or in vitro; d) increasing the viability of cells exposed to growth factors and/or mitogens and the therapeutic benefits derived therefrom, in vivo or in vitro; e) augmenting the biomass of cells, including stabilizing the cell(s) before, during, and/or after exposure to a proliferative stimulus and the therapeutic benefits derived therefrom, in vivo or in vitro; f) increasing lifespan of cells and the therapeutic benefits derived therefrom, in vivo or in vitro; g) enhancing the bioproductivity or function of cells, or both, and the therapeutic benefits derived therefrom, in vivo or in vitro; and h) increasing the spectrum of phenotypic expression available to cells, and the therapeutic benefits derived therefrom, in vivo or in vitro.

The lifespan of cells in culture is typically characterized in terms of population doubling level (PDL) of the cells, wherein each level represents a new generation of the cells. The time required for a population of cells to double is termed “generation time” (Tg), which varies with the growth stage of a given cell type. Under conventional culture conditions, each cell type has a lifespan characterized by a predictable number of population doubling levels, which are substantially the same for all healthy cells of a given type. Certain human cells, fibroblasts for example, under conventional culture conditions typically double in population from about 40 to 45 times before they senesce and stop normal growth; Tg increases, and death generally occurs at about PDL 50.

In accordance with one aspect of the present invention, the onset of senescence is delayed in cells by exposing these cells to one or more of the vitaletheine modulators described above. By the process of the invention, the population doubling level attainable by a given cell type before the onset of senescence and death increases significantly. At these high population doubling levels, the cell biomass is greatly increased, and the life expectancy of the cells is significantly extended; an increase from PDL 45 to PDL 105, for example, is achievable for human cells according to this process; this represents an increase in total cell mass as compared to biomass obtainable by conventional culture methods by a factor of 2⁶⁰. Further, the peak production period for cellular products is significantly prolonged, with optimization of other cellular functions. Additionally, the vitaletheine modulators are capable of eliciting enhanced cellular response to chemical, biochemical, or other stimuli, including the expression of functions different or additional, or both, to those expressed by the same type of cells at comparable stages of growth in vivo or under conventional culture conditions.

In order to rectangularize the life cycle of cells, e.g., optimize growth and maturation of cells and minimize the stages of senescence and death, it is preferred that the cells be exposed to the vitaletheine modulators before the onset of senescence. Since cellular aging is a gradual procedure, senescence may to some degree be arrested even if the cells are exposed to modulator at a later stage in the life of the cells, depending upon the particular cell type, and other factors. However, senescent cells are less viable and productive by definition, so maintaining them at this late stage of the lifespan is counterproductive for most aspects of the invention. Clearly, if the study of senescence is of primary concern then maintenance of the cells at this stage is of interest. Consequently, for optimum results in most instances, it is preferable to expose the cells to modulator as early in their life-cycle as is convenient.

Cells which are generally considered not amenable to culture are adapted to culture by exposure to adaptive amounts of the modulators. Included are cells which have a short lifespan under conventional culture conditions (e.g., from a few hours up to about a few weeks, for example, from about two hours to two weeks), or which do not function normally in culture (e.g., wherein in vivo cell bioproduction of hormones, enzymes, or other bioproducts is partially or substantially completely suppressed in vitro). Normal cells which do not in one or more respects exhibit in vivo behavior in culture, even under optimum culture conditions, as evidenced, for example by a foreshortened lifespan or abnormal cell function, are herein referred to as “resistant cells”. Such resistant cells are adaptable to culture by exposing the cells to be cultured to a vitaletheine modulator, ab initio, preferably by incorporating the modulator into the culture medium immediately before or soon after introduction of the cells, depending upon the particular culture medium and the stability of the particular vitaletheine modulator in that medium. Cellular function of resistant cells in culture is significantly improved, or substantially completely restored to normal cellular function characteristic of in vivo function, and/or cell lifespan is significantly improved or substantially completely restored to at least the cell lifespan characteristic of in vivo lifespans. Further, senescence of these cells is generally delayed in the presence of delaying amounts of modulators, often with a concomitant increase in, and potential diversification of, cellular function. Resistant cells include a variety of known resistant cell types, for example, lymphoid, hepatic, pancreatic, neural, thyroid, and thymus mammalian cells.

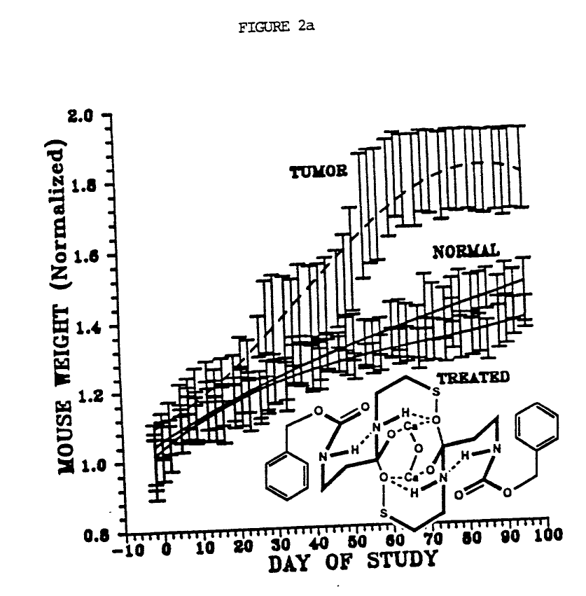

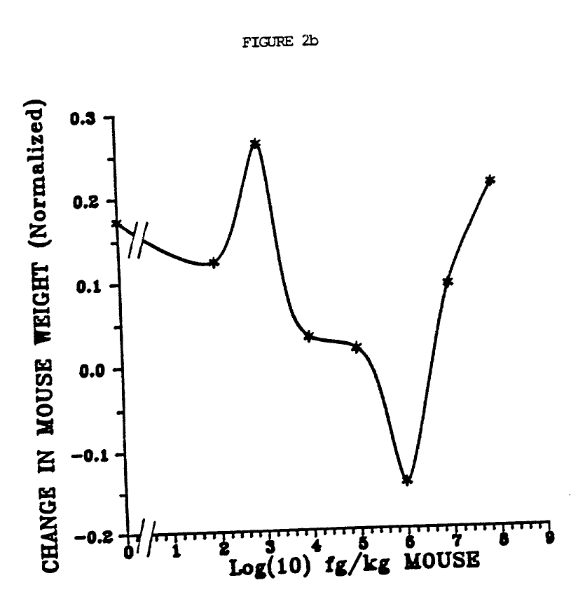

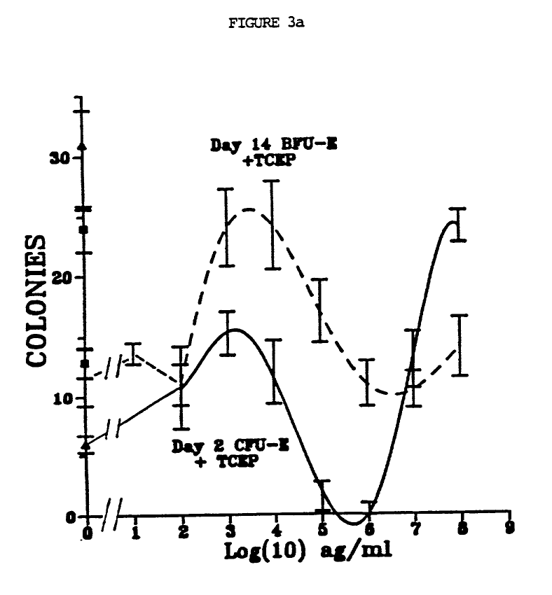

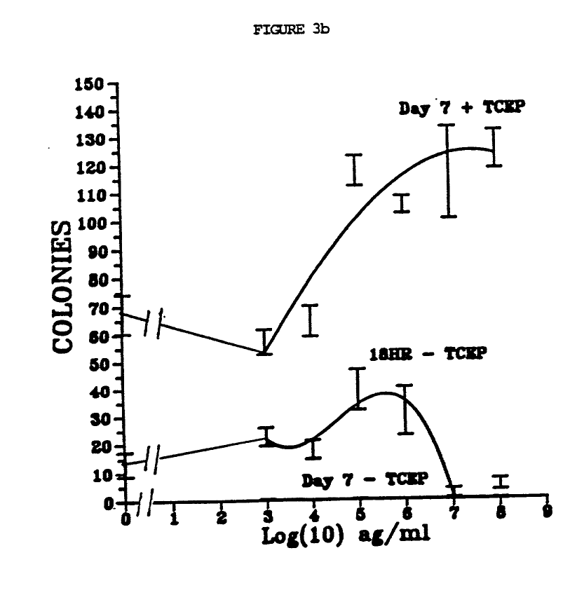

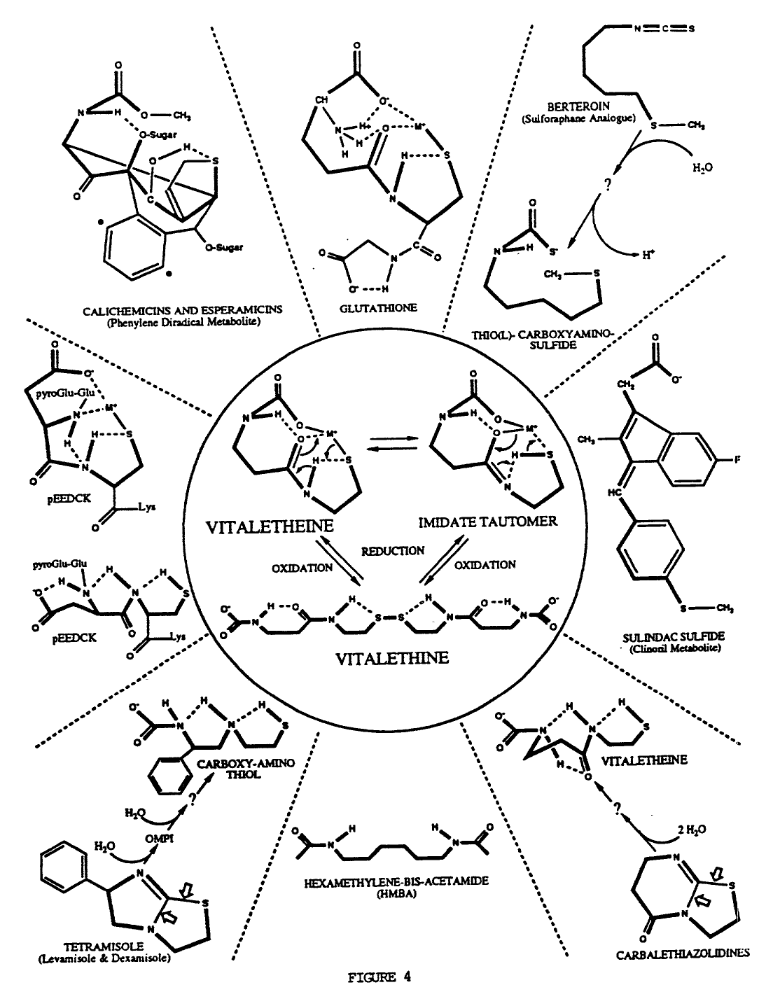

In vivo dosages for treatment of disease or disorder are conveniently determined by culture of the affected cells in vitro with exposure to the selected modulator in increments accompanied by measurement of cell bioproductivity to optimize dosage in vivo. Optimal in vitro dosages correlate well with in vitro dosages (compare FIGS. 2b and 3a), and are readily converted to in vivo dosages and regimens as described below. For modulators effective in the ag to fg/ml or fg to pg/kg ranges of concentrations direct conversion is possible if selected cells are targeted. When these modulators are uniformly distributed, up to about a thousand fold increase in in vivo concentrations are necessary to accomodate the higher density of cells in vivo than in cell culture.

Culture media in which vitaletheine modulators are to be incorporated for modulation of cell activity of cells cultured therein do not form a part of the invention. Exemplary useful media include all known culture media and media hereinafter developed which support maintenance and/or growth of the cells therein cultured. Such media typically comprise at least nutrients suitable for the growth of the specific cells to be cultured, a physiological balance of electrolytes, a physiological pH, and water, as necessary to support cell growth, as well as physical culture aids such as cell supports. A variety of other known auxiliaries such as antibiotics, sera, or cell growth regulators may also be included in the basal culture media into which the modulators are to be incorporated, especially those known for enhancing cell propagation, or for augmenting cell growth and/or longevity, including cell growth factors such as peptidyl hormones specific for the cells being cultured, of the type well-known in the art. These and other auxiliaries which affect cell longevity and function in some respects are optionally included in the basal culture medium providing that they do not completely obviate the activity of the vitaletheine modulators; in fact, selective proliferation with one or more of these factors, such as, for example, specific peptidyl hormones, in the presence of a vitaletheine modulator to stabilize the cells being generated comprises a useful technique for selectively enriching the cells of interest in a gross cellular extract, for example, organ extracts. Compounds which inhibit metabolism of the modulators may also be included.

Conventional media into which the modulators are incorporated for the practice of the invention are herein referred to as “basal culture media”. Basal culture media into which the modulators are incorporated may -be employed in conjunction with any suitable culture techniques known or hereinafter to be developed, including batch or continuous culture, perfusion culture, or other techniques, particularly those adapted to maximize cell culture, as by the continuous replenishment of nutrients or other media components and continuous removal of cell waste materials.

Broadly, the modulators are suitable for modulating the activity of cells in any medium which does not significantly inactivate or otherwise adversely affect the function of the modulators, and which optionally supports the growth of these cells.

The cells may be exposed to the modulators in any convenient fashion. The modulators may, for example, be incorporated into the nutrient medium, or into cell support elements. The cells may also be pre-exposed to modulator. The modulators are incorporated into a support material by combining the modulators with starting materials employed to prepare the supports. Introduction of modulators into synthetic prepolymers for the production of natural or synthetic supports such as hollow fiber membranes, or pregels for the production of gel supports, or liquefied cellulose for the production of cellulose supports, are exemplary. The modulators may be injected in any convenient physiologically acceptable vehicle, including saline and phosphate-buffered saline, in any location which does not result in the irreversible inactivation or maladsorption of the modulator, including i.v., i.p., s.c., and i.d. Likewise, the modulators may be administered in any other fashion which does not result in the irreversible inactivation or maladsorption, such as orally with the appropriate additionally optional entero-coating, rectally, nasally including sprays, and dermally including patches.

Culture media employable with the modulators include known basal media optionally supplemented with protein components, particularly serum, e.g., fetal or new-born calf serum. Exemplary media include Eagle's Basal Medium; Eagle's Minimal Essential Medium; Dulbecco's Modified Eagle's Medium; Ham's Media, e.g., F10 Medium; F12 Medium; Puck's N15 Medium, Puck's N16 Medium; Waymoth's MB 7521 Medium; McCoy's 5A Medium; RPMI Media 1603, 1634, and 1640; Leibovitz's L15 Medium; ATCC (American Type Culture Collection) CRCM 30; MCDB Media 101, 102, 103, 104; CMRL Media 1066, 1415, 1066, 1415; and Hank's or Earl's Balanced Salt Solution. The basal medium employed, as known in the art, contains nutrients essential for supporting growth of the cell under culture, commonly including essential amino acids, fatty acids, and carbohydrates. The media typically include additional essential ingredients such as vitamins, cofactors, trace elements, and salts in assimilable quantities. Other biological compounds necessary for the survival/function of the particular cells, such as hormones and antibiotics are also typically included. The media also generally include buffers, pH adjusters, pH indicators, and the like.

Media containing the modulator or modulators are applicable to a variety of cells, especially eukaryotic cells. The media are suitable for culturing animal cells, especially mammalian cells; plant cells; insect cells; arachnid cells; and microorganisms such as bacteria, fungi, molds, protozoa, and rickettsia, especially antibiotic-producing cells.

The modulators are broadly useful to promote viability of living cells in a broad spectrum of so-called tissue culture media adapted for the culture of such cells. Exemplary applications include the culture of cloned cells, such as hybridoma cell lines; of mammalian cells for the production of cell products, especially proteins and peptides such as hormones, enzymes, and immunofactors; of virally-infected cells for the production of vaccines; of plant cells in, for example, meristem or callus culture; of epithelial cells to provide tissue for wound healing; of resistant cells for medical and diagnostic use; and in media adapted for the production and preservation of biological organs and implant tissue.

Culture techniques useful in conjunction with the modulators include the use of solid supports, (especially for anchorage-dependent cells in, for example, monolayer or suspension culture) such as glass, carbon, cellulose, hollow fiber membranes, suspendable particulate membranes, and solid substrate forms, such as agarose gels, wherein the compound is caged within the bead, trapped within the matrix, or covalently attached, i.e. as a mixed disulfide. The modulators are useful in primary cultures; serial cultures; subcultures; preservation of cultures, such as frozen or dried cultures; and encapsulated cells; cultures also may be transferred from conventional media to media containing the modulators by known transfer techniques.

According to the practice of the invention, cells are exposed to one or more active vitaletheine modulators, or one or more active or activatable modulators, of the Formulas I through X in an amount sufficient to promote the desired biological response of these cells in vitro or in vivo, as measured, for example, by significant increase in cell lifespan or viability, increase in cell biomass, increase in cell bioproductivity, delay of cell senescence, diversification or normalization of cell function, or elimination of intractable cells, as compared to unexposed cells. Modulators which delay or obviate disease, normalize aberrant cell behavior, and/or eliminate intractable cells are of particular interest.

Modulators useful for modulating cells in vitro or in vivo according to the invention comprise active- or activatable-compounds of the Formulas I through X. As used herein, “active vitaletheine modulators” comprise compounds of the Formulas I through X which per se modulate cells in vivo or in vitro; those which directly delay or obviate disease, normalize aberrant cell behavior, and/or eliminate intractable cells are of particular interest. The term “activatable vitaletheine modulators” as used herein refers to compounds of the Formulas I through X which are not in themselves active, but are activatable to compounds which similarly modulate cells in vitro or in vivo, especially those which directly delay or obviate disease, normalize aberrant cell behavior, and/or eliminate intractable cells, primarily by rearrangement including reversible cyclization and tautomerization, dehydration, hydration, salt exchange, oxidation, and/or reduction of the compounds as described herein, either before the modulators are incorporated in the culture medium, or by appropriate adjustment of the culture medium, for example with regard to pH, salt, partial pressure of O₂ or CO₂, enzyme content, exposure to UV or other radiation, and temperature. The characterization of a given modulator as either “active” or “activatable” for a particular application is dependent on a variety of factors, including environment of the cell and cell type, and selection of modulators for optimum results is made accordingly.

In practice, it is generally preferred to employ naturally-occurring vitaletheine modulators of the Formula II and subformulas thereof, as the derivatives thereof of the Formula III et. seq. are not believed to be endogenous compounds and their metabolic pathways are at present unknown. The naturally-occurring modulators of the Formula II are postulated to be endogenous to a broad spectrum of cells, including animal, plant, insect, arachnid, and microorganism cells, and accordingly, most, if not all, cells derived from these organisms are expected to have well-established mechanisms for the enzymatic activation, utilization, and metabolism of these compounds. Thus, to maximize efficacy and minimize potentially toxic or undesirable side reactions, the use of either naturally-occurring modulators of the Formula I or vitaletheine modulators activatable to the naturally-occurring modulators in the practice of the invention is recommended, especially vitalethine, vitaletheine, or vitaletheine V₄ of the Formulas IId, IIe, and IX.

The use of modulators according to the present invention in modulating cells in vivo or in vitro, especially by delaying or obviating disease, normalizing aberrant cell behavior, and/or eliminating intractable cells, is contemplated to be applicable to the broad range of cells recited, owing to the postulated near-universality of precursors to the compounds of the Formula II in the metabolic pathways of at least eukaryotic organisms, and the biochemical equivalence of the non-naturally occurring homologs and analogs of Formulas III through VIII.

The effect of the modulators on cellular growth patterns is typically concentration-dependent. Optimization of efficacy, especially with respect to cell life expectancy and maximization of cell function (e.g., rate of bioproduction and/or diversity or normalization of function) may occur within a relatively narrow concentration range of modulator; outside this range, cell growth patterns and/or cell functions may tend to approach those of untreated cells. Also, the process of the invention may be, at least in some instances, reversible; that is, cells retained in culture by exposure to the modulators beyond their normal lifespan may, for example, revert to the senescent patterns of untreated cells soon after failure to properly replenish the modulator.

The amount of modulator eliciting the desired biological response according to the present invention is herein referred to as an “effective amount” of modulator. Optimum amounts of modulator for delaying senescence, herein referred to as “senescence-delaying” amounts, are readily determined by introducing varying amounts of modulator into test cultures substantially before the onset of senescence, and selecting the concentration at which the lifespan of cells in culture is maximized. As previously noted, an amount of modulator sufficient to increase, for example, a selected cell function is often substantially equivalent to the amount of modulator required to effect other modulations of cell activity. Since this may not always be the case, it is useful to adjust modulator concentration against the specifically desired end result; for example, improved rate of cell bioproduction, improved span of cellular bioproduction, improved diversity of cellular function, improved delay or obviation of disease, or improved life expectancy of cells.

As a general guideline for effective concentrations of modulator for promoting cell production according to the invention, especially for promoting cellular phenotypic expression, function, and viability, delaying senescence, promoting adaptation of cells to culture, and particularly for delaying or obviating disease, normalizing aberrant cell behavior, and/or eliminating intractable cells, from about 0.01 fg to 100 ng vitaletheine modulator(s) per milliliter culture, and preferably from about 0.1 to 10,000 fg vitaletheine modulator(s) per milliliter culture is recommended, or for in vivo applications from about 0.1 fg to 1,000 ng vitaletheine modulator(s) per kg body weight, and preferably from about 1 fg to 10 ng vitaletheine modulator(s) per kg body weight is recommended, depending particularly on the potency of the modulator and cell densities. When combinations of the modulators are employed, total amount of modulator will usually be within these ranges. Since the effective amount at the lower concentrations of vitaletheine modulator(s) recited approaches one molecule of modulator per cell, it is especially important to adjust the concentration of modulator at the lower end of these ranges according to the number of cells present, i.e., the cell density. Most preferably, the basal culture medium employed is supplemented with sufficient modulator to provide a total concentration of modulator(s) in the medium of from about 1 to 2 fg modulator per milliliter of medium, again depending primarily upon the potency of the modulator, the type of cell, and upon cell densities. Likewise, for in vivo applications total concentration of modulator(s) is most preferably from 10 fg to 100 pg/kg depending upon the potency of the modulator, the type of cell, and upon cell densities. Typically, the above concentration ranges of modulator(s) will comprise effective amounts of modulator for cultures irrespective of cell densities, but special problems of nutrient and modulator supply and waste removal exist in confluent cultures. Consequently, confluent cultures should be avoided when possible unless special provisions are made for these environmental needs. Up to ten million cells per milliliter culture is a useful range of cell concentration, for confluency increases at higher cellular densities, again depending upon the size of the cells. Typical cell densities comprise from about one hundred thousand to ten million cells per milliliter culture, and the above described dosages are based upon such densities. Since the effective concentration of modulator has approached one molecule per cell, the concentration of modulator is varied as the concentration of cells increases or decreases.

Replenishment of the vitaletheine modulator(s) to regulate cell activity as desired may be advisable. Diurnal variations in enzymatic activity are notable, and diurnal or 48 hour replacement is generally recommended, typically depending upon the stability of a particular vitaletheine modulator(s) in the particular environment and the particular type of cell employed. There is some evidence that the compounds accumulate with prolonged treatment regimens, in which case it is advisable to diminish the concentrations administered.

Based on illustrated and non-illustrated research data, it appears that cells to be modulated according to the invention may demonstrate an inherent resistance to extra-biological amounts of vitaletheine modulator(s). This is overcome as concentration(s) are increased at a dosage at which a response is first observed, herein referred to as “threshold dosage”. The response augments rapidly with dose to a maximum response at a dosage herein referred to as “optimum dosage”; beyond this point, the cell response typically declines with increasing dose to that observed in unexposed cells. The dosage at which basal biological activity is restored is referred to herein as “endpoint dosage”. The dosage providing a response from between about the threshold dosage and the endpoint dosage is referred to herein as the “effective concentration or dosage” of the modulator.

Guidelines for the development of dose-response curves for a particular application are conveniently developed as follows:

### DOSE RESPONSE CURVE DEVELOPMENT GUIDELINES

A. Employing Vitaletheine Modulator(s) for Delaying Senescence.

Cells of the type to be cultured according to the process of the invention are first grown in a modulator-free control basal culture medium according to standard practice to measure generation time. The onset of senescence is marked by a significant increase in the cell generation time, as is well-understood in the art. Samples of the same cell type at chronologically identical stages of development are then cultured in the same medium containing a modulator in the amounts ranging for example from about 0.1 femtograms vitaletheine modulator(s) per milliliter to about 1 microgram vitaletheine modulator(s) per milliliter culture medium, based on exemplary cell densities of about one million cells per milliliter culture; preferably, dose of the compound in log₍₁₀₎ increments are used to localize the effective concentration of any particular vitaletheine modulator. The cultures are then reexamined over a range flanking the effective dosage in less than one log(₁₀₎ increments to thoroughly define the effective concentration, the threshold dosage, and the endpoint dosage for that particular culture.

Up to a doubling of the normal lifespan and/or presenescent life of cells in culture is commonly observable according to the process of the invention, and in many instances three-fold or more increases in lifespan are obtainable. Further, cells cultured according to the present process exhibit differences in phenotypic expression, thought to be more characteristic of the cells, in vivo, as compared to untreated cells.

B. Employing Vitaletheine Modulator(s) in in vivo Applications.

Biological activities to be modulated according to the invention are generally evaluated in parallel in the presence and absence of vitaletheine modulator(s) to establish the basal biological activities under conditions identical to the evaluation of modulated activities. Modulators are administered by routes previously described while the control or basal group receives only the vehicle for administration. For example, groups of 5 or more animals are treated with log₍₁₀₎ increments from 0.1 fg to 1,000 ng vitaletheine modulator(s)/kg body weight (such as by i.p. injection in physiological saline, optionally including inhibitors of the metabolism of the modulator(s)), periodically throughout the study, such as three times per week. Samples or measurements are taken and preserved as the regimen is continued for at least two weeks and preferably for 15 weeks. After compilation of the data, the response is evaluated graphically with a three dimensional surface in which the X, Y, and Z axes are dose, week, and response, respectively. The optimum concentration of modulator is easily identified in this manner; and if inhibitor(s) of metabolism have been included, the study is repeated holding this concentration constant and varying the concentration of the inhibitor(s) to optimize the inhibitor(s) concentration(s) as well. Repeating the analysis with more closely spaced increments of modulator and with a constant optimal dose of inhibitor, then, localizes the optimum and effective range of concentrations for compounds.

Therapeutic effects are obtainable with as little as 100 ng of inhibitor/kg body weight and with less than 100 pg vitaletheine modulator/kg body weight.

### EXAMPLES

### Example I

### Synthesis of N,N′-bis-(CBZ)-β-alethine {S,S′-Bis[(N-carbobenzoxy-β-alanyl)-2-aminoethyl] Disulfide}

A solution of dicyclohexylcarbodiimide (23.3 g) was added to a solution of N-CBZ-β-alanine (24.84 g) and N-hydroxysuccinimide (12.92 g) in a total volume of about 500 ml of dry 10% acetonitrile in dichloromethane. Dicyclohexylurea (24.51 g) precipitated as a by-product upon formation of the active ester. The active ester was dried to an oil and triturated with anhydrous ethyl ether. The precipitate was resuspended in dichloromethane and additional dicyclohexylurea was allowed to precipitate. The resulting dichloromethane solution of active ester was filtered and added to a previously prepared solution of cystamine (8.5 g). The desired product, N,N′-bis-(CBZ)-β-alethine precipitated from this mixture. The mother liquor, anhydrous ether and dichloromethane extracts of the product, and the anhydrous ether extract of the active ester, above, were dried and recombined to augment the yield of product. N,N′-bis-(CBZ)-β-alethine was practically insoluble in water, hot ethyl acetate, and hot ether, and these were used to further extract impurities. The product was recrystallized from dimethyl sulfoxide with acetonitrile (or water), and again rinsed with ethyl acetate and ether. This last process resulted in a 1° C. increase in melting point to 180-181° C. (uncorrected). Yields of N,N′-bis-(CBZ)-β-alethine of 85-90% were routinely obtained, and near-quantitative yields are possible. When dried over P₂O₅, in vacuo, the product appeared to retain one mole equivalent of water, and was analyzed accordingly as the monohydrate.

Anal. Calcd. for C₂₆H₃₄N₄O₆S₂.H₂O: C, 53.78; H, 6.25; N, 9.65. Found: C, 54.23; H, 6.56; N, 9.66. Sample analyzed by Ruby Ju, Department of Chemistry, University of New Mexico, Albuquerque, N. Mex.

### Example II

### Synthesis and Characterization of the Benzyl Derivative of Vitaletheine

A. Synthesis. The following reagents were added with mixing in the order listed to an Erlenmeyer flask (500 ml): N,N′-bis-(carbobenzoxy)-β-alethine (0.76 g) from Example I, above, dimethyl sulfoxide (0.75 ml), N,N′-dimethylformamide (0.75 ml), pyridine (1 ml), chloroform (21 ml), water (150 ml), and iodine (3.3 g). Upon addition of the iodine the pH began to decrease, and was maintained at 5.7 by slowly adding zinc oxide (0.3 to 0.4 g). It was desirable to maintain this slightly acidic pH to optimize reaction rates. This mixture allowed controlled reaction, continuous extraction of the intermediate product from the organic reagent phase into the aqueous phase, and continuous monitoring of the pH of the aqueous phase. When the reaction began to subside, which was indicated by a stabilization of pH, the aqueous phase was removed and subjected to repeated extractions with chloroform until no color was evident in the organic phase. Periodically during these extractions, the pH was readjusted to 6.0 with a minimum amount of ZnO. When completely extracted and neutralized to pH 6.0, the aqueous phase was dried on a rotoevaporator at low temperature (<40° C.) to a viscous oil. During this process, the organic phase of the reaction mixture was reextracted with water to recover residual intermediate product, which was subsequently extracted with chloroform, neutralized with ZnO, and dried with the first aqueous extract.

This stage in the synthesis represents a branch point for the synthesis of the desired compound; at this point, either the desired compound or the benzyl derivative thereof can be obtained. For example, either vitaletheine V₄ (Example III and Formula IX) or the benzyl derivative of vitaletheine of the Formula VIII can be produced at this stage.

To obtain the benzyl derivative of vitaletheine, the aqueous extracts obtained as above were treated with ten volumes of acetonitrile to precipitate the benzyl derivative as the primary product.

B. Characterization of the Benzyl Derivative of Vitaletheine The benzyl derivative obtained above had approximately the same molecular weight as the blocked alethine starting material. However the derivative was unlike N,N′-bis-(CBZ)-β-alethine in many respects: it was soluble in water; it had unique [¹³C]- and [¹H]-NMR spectra; and its IR spectrum was likewise distinct. The benzyl derivative was purified as the calcium salt, but this difference from the zinc salt of vitaletheine V₄ (below) could not account for the extremely high melting point of the former; the benzyl derivative melted at temperatures in excess of 300° C., while the starting material melted at 180-181° C. (uncorrected). The NMR spectra of the zinc and calcium salts of the benzyl derivative were quite similar, evidence that salts alone could not account for these differences.

The spectra of the benzyl derivative were not consistent with thiazolidine or cyclic-urethane structures, and no detectable disulfide or thiol was present, suggesting that like vitaletheine V₄, the benzyl derivative was formed by the nucleophilic attack involving sulfur on one of the carbonyl carbons in each monomer. Unlike vitaletheine V₄, the predominant polymer in the product benzyl derivative was identified as a dimer, probably formed by attacks of each monomer on the carbonyl carbon of the other, as described above. The quaternary carbons present appeared identical, and were not shifted upfield (**) in the NMR spectrum, in contrast to the pronounced upfield shift of the quaternary carbon atoms present in the vitaletheine tetramer, indicating fewer structural constraints in the benzyl derivative dimer than in the vitaletheine tetramer. Elemental analysis indicated additional material had crystallized with the benzyl derivative, and good correlation was found for inclusion in the dimer of 2 mole equivalents of calcium ion and one mole equivalent of oxygen per mole of the dimer. This was consistent with the presence of a calcium oxide bridge between two dimers, stabilized by hydrogen bonding. The following was the result of elemental analysis for the benzyl derivative obtained above, correcting for the presence of the calculated oxygen and calcium ion:

Anal. Calcd. for C₂₆H₃₄N₄,O,S₂2 Ca⁺⁺O⁼: C, 45.20; H, 4.96; N, 8.11. Found: C, 44.97; H, 4.98; N, 8.04. Sample analyzed by Ruby Ju, Department of Chemistry, University of New Mexico, Albuquerque, N. Mex.

### Example III

### Synthesis and Characterization of Vitaletheine V₄

A. Synthesis. The benzyl group was removed by repeatedly exposing the dried aqueous extracts obtained in Example IIA to ultraviolet light (Pen-ray quartz lamp, Ultra Violet Products, Inc., Cambridge, U.K.) and extracting with chloroform until no color developed under UV irradiation, and no color was extractable into chloroform. UV irradiation is particularly recommended for effectively obtaining product substantially devoid of aromatic moieties, without causing more serious and inactivating rearrangements and decompositions, as described previously. The product (when completely free of aromatics) was dried, neutralized in water with ZnO, and recrystallized from dimethylsulfoxide with acetonitrile to yield the zinc salt of vitaletheine V₄.

B. Characterization of Vitaletheine V₄. Vitaletheine V₄ was likewise distinct with reference to both the starting material and the benzyl derivative. Obtained in greater than 50% yield from the above procedure, it melted with decomposition at 233-235° C. (uncorrected). Evolution of gas signified decomposition of the molecule; the evolved gas (CO₂) was trapped by bubbling through a saturated solution of Ba(OH)₂ under N₂, with recovery of BaCO₃. Decomposition of the molecule on heating was consistent with the presumptive thermal lability of the postulated carboxyamino structure, as was the evolution of CO₂ upon heating, and the recovery of the trapped CO₂ as the insoluble barium carbonate. The possibility that the evolved gas resulted from decomposition of zinc carbonate contaminating the vitaletheine V₄ was deemed unlikely, since this salt decomposes with CO₂ evolution at 300° C. The spectral evidence likewise indicated a structure unique to vitaletheine V₄, comprising covalent attachment of the carbon in question (2) to the β-aletheine moiety. Concomitant with the evolution of CO₂, losses of a sharp N-H stretch resonance at 3290 cm⁻¹ and other resonances associated with the carboxyamino structure were observed in the IR spectra.

Vitaletheine V₄ as prepared was somewhat hygroscopic, possibly exacerbated by residual dimethylsulfoxide. The following elemental analysis reflected the propensity of the molecule to gain water:

Anal. Calcd. for C₂₄H₄₄N₈O₁₂S₄.2 Zn⁺⁺.8 H₂O: C, 27.72; H, 5.82; N, 10.78. Found: C, 28.56; H, 5.94; N, 10.96. Sample analyzed by Ruby Ju, Department of Chemistry, University of New Mexico, Albuquerque, N. Mex.

The results of several different analyses indicated that the vitaletheine dimer contained 1 Zn⁺¹, the trimer contained 1.5 Zn⁺², and the tetramer contained 2 Zn⁺² per mole of polymer.

### Example IV

### Synthesis and Characterization of Vitalethine via β-alethine.

A. Synthesis of β-alethine.2HCl or N,N′-bis-(β-alanyl)-cystamine or N,N′-bis-(β-alanyl-2-aminoethyl) disulfide. Complete removal of the carbobenzoxy group was accomplished according to procedures described in J. Am. Chem. Soc. 86: 1202-1206 (1964), incorporated herein by reference. After deblocking with four equivalents of hydrogen bromide in glacial acetic acid per mole of the N,N′-bis-(CBZ)-β-alethine (from Example I, above) for 15 hours, the β-alethine was purified by precipitating with acetonitrile, rinsing with anhydrous ethyl ether, resuspension in water and filtering, and precipitating the mixed salts with acetonitrile. Initial yields were in excess of 80% theoretical. The β-alethine was converted to the hydrochloride salt by passing the preparation over a 30 ml×15 cm long column of Dowex AG 1×8 (chloride form) (Dow Chemical Corp., Midland, Mich., USA) which had been previously prepared by eluting with 1 M potassium chloride and rinsing thoroughly with DI (deionized) water. Neutralization with Ca(OH)₂ and recrystallization of the β-alethine hydrochloride from water with acetonitrile resulted in fine needles which melted at 224-225° C. (uncorrected).

Anal. Calcd. for C₁₀H₂₂N₄O₂S₂.2HCl: C, 32.69; H, 6.59; N, 15.25. Found: C, 32.52; H, 6.69; N, 15.32. Sample analyzed by Ruby Ju, Department of Chemistry, University of New Mexico, Albuquerque, N. Mex.

B. Synthesis of Vitalethine. To a suspension of ZnO (6.5 mg from King's Specialty Company, Fort Wayne, Ind., USA) and β-alethine (6.35 mg from Example IV. A. above) in pyridine (12.6 mg from Fisher Scientific, Fair Lawn, N.J., USA) and dimethylsulfoxide (0.5 ml from Sigma Chemical Company, St. Louis, Mo., USA), and in a vessel equipped with a gas trap containing sodium hydroxide (at least 1M), was added 0.2 ml of a solution of phosgene (20% in toluene from Fluka Chemical Corp, Ronkonkoma, N.Y., USA). Packing the reaction vessel in dry ice controls the exothermic reaction and improves yields of large-scale preparations. After 48 hours of reaction the excess phosgene was blown into the alkali trap with N₂. The product was precipitated in the vessel with acetonitrile (approximately 50 ml from Fisher Scientific, Fair Lawn, N.J., USA). Vitalethine can be recrystallized from water with acetonitrile.

C. Characterization of Vitalethine. Unlike the starting material, β-alethine which melted at 224-225° C. (uncorrected), the vitalethine powder sintered and turned brown at 215-220° C., but did not melt until 242° C. (uncorrected) at which point obvious decomposition and evolution of gas occurred. This behavior resembled that of vitaletheine V₄, in that gas was also evolved upon melting of the polymer. The infrared spectrum of the two compounds were likewise similar, but the vitalethine spectrum did not exhibit the C—O stretch bands observed in the polymer. Both compounds lost infrared resonances associated with the carboxy-amino group upon thermally labilizing this moiety. This was particularly true of vitalethine, for major peaks disappeared at 1600 and 1455 CM⁻¹ (resonances for the ionized carboxylic moiety), and losses in the fine structure in the regions 2800 to 3300 CM⁻¹ and 900 to 1360 CM⁻¹ (i.e., those associated with the N—H and C—N moieties of the carboxy-amino group) were also apparent upon heating at 242° C.

### Example V

### [¹³C]-NMR, [¹H]-NMR, and IR Spectra of Vitalethine, V₄ and Related Compounds

| | | | | |
|---|---|---|---|---|
| a S—CH₂ | b CH₂—N | c H—N—C═O | d O═C—CH₂ | e CH₂—N |

[¹³C]-NMR

| | | | | | | |
|---|---|---|---|---|---|---|
| β-alethine | 37.59 | 39.04 | 172.79 | 32.9 | 36.71 | |
| Vitaletheine | 36.66 | 35.93 | 47.06*44.75 | 50.39 | 32.96 | 172.73 |
| V₄ | | | 39.41*38.51 | | | |
| Benzyl | 33.79 | 35.76 | 154.46** | 48.36 | 34.67 | 172.25 |
| derivative | | | | | | |

[¹H]-NMR

| β-alethine* | 2.524 | 3.094 | | 2.694 | 3.367 | |
|---|---|---|---|---|---|---|
| β-aletheine | 2.512 | 3.084 | | 2.695 | 3.372 | |
| (Zn++) | | | | | | |
| β-aletheine | 2.512 | 3.087 | | 2.687 | 3.366 | |
| (+I₂) | | | | | | |
| Vitaletheine | | | | | | |
| V₄ (D₂O) | 2.502 | 3.081 | | 2.937 | 3.416 | |
| (DMSO-D6) | 2.200 | 2.763 | 7.84 | 2.418 | 3.131 | 7.38 |
| Benzyl | | | | | | |
| derivative | | | | | | |
| (D₂O) | 2.232 | 3.201 | | 2.841 | 3.330 | |
| (DMSO-D6) | 2.210 | 3.176 | 7.84 | 2.593 | 3.309 | 7.247 |
| bis-(CBZ)- | 2.740 | 3.309 | 8.085 | 2.254 | 3.192 | 7.24 |
| β-alethine | | | | | | |
| (DMSO-D6) | | | | | | |
| Reductase | 2.71 | 3.08 | | 2.90 | 3.28 | |
| Factor | | | | | | |
| (Inactive) | | | | | | |
| | S—CH₂a | CH₂—N b | H—N—C═O c | O═C—CH₂d | CH₂—N e | 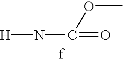 |

*β-alethine was reduced with REDUCTACRYL* (a proprietary reducing agent available from Calbiochem, San Diego, Calif., USA) in the presence of ZnO to form β-aletheine. The latter reacted with I₂ to provide a third reference compound, probably the sulfenyl iodide.

IR (cm-1)

| | a S—CH₂ | b CH₂—N | c H—N—C═O | d O═C—CH₂ | e CH₂—N | 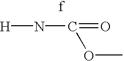 |
|---|---|---|---|---|---|---|
| Vitalethine | | | 3170 w | | | 3290 m |
| | | | 1550 w | | | 1560 s |
| | | | | | | 1600 m |
| | | | | | | 1455 s |
| Vitaletheine | 710 w | 3080 s | | | 3290 s | |
| V₄ | | | 1530 m | | | 1560 s |
| | | | | | | 1253 m |
| | | | | | | 1650 s |
| | | | | | | 956 m |
| Benzyl | 692-570 w | | 3308 s | | | 3308 s |
| derivative | | | 1542 s | | | 1542 s |
| | | | 1635 s | | | 1253 m |
| | | | | | | 1684 s |
| bis-(CBZ)- | | | 3345 s | | | 3345 s |
| β-alethine | | | 1545 m | | | 1535 s |
| | | | 1640 s | | | 1270 m |
| | | | | | | 1682 s |
| | a | b | c | d | e | f |
| | S—CH₂ | CH₂—N | H—N—C═O | O═C—CH₂ | CH₂—N | —N—H |
| β-alethine | 660 w | | 3250 w | | | 3270 v |
| | | | 1555 w-s | | | 2970 s-w |
| | | | 1286 m | | | 1462 s |
| | | | 1620 s | | | 1620 s |
| | | | | | | 1128 s |

Vitaletheine V₄ and vitalethine were unique in that resonances associated with the moiety “f” above disappeared when the compounds melted and decomposed at 233-235° C. (uncorrected) and 242° C., respectively, presumably due to loss of CO₂. In vitaletheine V₄, these losses occurred without concomitant losses in the regions designating a (—C—O—)_{y} polymer; thus the decarboxylated form of Vitaletheine V₄ appeared to be an oligomer of β-aletheine similar to the undecarboxylated polymer, but lacking the carboxy moieties.

Peaks for Vitalethine: 3290m, 3170w with shoulder at 3100, 2990m, 1660s, 1600w, 1565m, 1455s, 1410w with 1400 shoulder, 1330w with 1310 shoulder, 1260m with 1230 shoulder, 1190w, 1135m, 1100m with 1090 shoulder, 1030m-s, 955m.

Peaks for heated Vitalethine: 3120s (broad), 1655s, 1550m, 1405s with shoulders at 1450 and 1390.

The IR spectrum of vitaletheine V₄, following, was shifted by exchanging acetonitrile for water of hydration in the complex.

Peaks for Vitaletheine V₄: 3290s, 3080s/broad to 2500, 1650s, 1560s, 1530m, 1453w, 1417w, 1393w, 1346w, 1318w, 1253m, 1190s, 1170s, 1115w/shoulder, 1040s, 1030s, 956m, 790m with shoulder, 709w/broad, 612m/sharp, 526m. These shifts approximated those observed in the spectrum of β-alethine upon neutralization, below.

β-alethine was unusual in that changes in pH, i.e., neutralization with Ca(OH)₂, caused pronounced shifts in the positions and intensities of bands.

Peaks (HCl salt): 3270s, 3170s, 2970s, 2700w, 2550w, 2020w, 1657s, 1595m, 1560s, 1450s, 1409m, 1390w, 1354w, 1325m, 1300w, shoulder/1252m/shoulder, 1188m, 1129m, 1097m, 1079w, 1030w, 950w, 905w, 829m.

Peaks (neutralized): 3250w, 3180w, 2940m/broad, 2375s, 2230s, 2157s, 1936w, 1620s, 1555w, 1462s, 1432 shoulder, 1400m, 1342m, 1286m, 1217m, 1188m, 1128s, 1020m, 810w, 719m, 660w.

The benzyl derivative displayed considerable homology with vitaletheine V₄.

Peaks: 3308s, 3060w, 2942w, 1684s, 1635s, 1542s, 1447m, 1380w, 1335w, 1286w, 1253m, 1193s, 1170 shoulder, 1080m, 1040m, 980w, 738m, 692m, 609m, 550w.

Bis-(CBZ)-β-alethine displayed little of the C—O resonances around 1200 observed in vitaletheine V₄ and the benzyl derivative.

Peaks: 3345s, 3310s, 1682s, 1640s, 1545m shoulder, 1535s, 1450w, 1427w, 1375w, 1332m, 1270m, 1231m, 1178w, 1120w, 1030m/broad.

In the following Examples, all cells were cultured at about 37° C. for the specified time.

### Example VI

### Adaptation of Human Natural Killer (NK) Cells to Culture

Human NK cells were purified as described in J. Exp. Med. 169: 99-113, 1989. A standard culture medium for the cells was prepared, comprising RPMI 1640 (Rosewell Park Memorial Institute, from Whittaker M.A. Bioproducts, Walkersville, Md., USA) containing 10% human AB-sera, penicillin (100 U/ml) and streptomycin (100 μg/ml), which served as the control medium. Experimental media were prepared by adding 25 μl/ml of an appropriate aqueous dilution of vitaletheine V₄ to obtain the following final concentrations in separate aliquots of medium containing cells otherwise identical with the controls: 0.1 fg/ml, 1 fg/ml, 10 fg/ml, 100 fg/ml, 1 pg/ml, and 10 pg/ml.

Purified cells (1×10⁶) were seeded and incubated in the control and test media at 37° C. under 5% CO₂. Cells were counted, and checked for viability daily by monitoring trypan blue (0.1% in phosphate buffered saline) exclusion, and the media containing the same vitaletheine V₄ concentration were changed every two days to maintain physiological pH and to remove waste products from the cells.

Dramatic stabilization of the NK cell population in culture was observed with vitaletheine V₄. By day five, no cells survived in the unsupplemented, i.e., control medium. In media containing vitaletheine V₄, 70 to 80% of the cells survived for more than a week. Although the extremes of the effective concentration were not defined in this particular experiment, two doses of vitaletheine V₄ were selected for further study.

The results of the viability tests are summarized in Table I, following:

TABLE 1

| Day | No V₄ | 1 fg V₄/ml | 1 pg V₄/ml |
|---|---|---|---|
| 0 | 98 ± 2 | 98 ± 2 | 99 ± 2 |
| 1 | 96 ± 1.5 | 98 ± 2 | 99 ± 2.5 |
| 2 | 45 ± 1.8 | 97 ± 1.5 | 98 ± 3 |
| 3 | 30 ± 1.5 | 98 ± 2.5 | 98 ± 2 |
| 4 | 15 ± 0.5 | 97 ± 3 | 97 ± 3 |
| 5-20 | 0 ± 0 | 97 ± 3 | 97 ± 3 |

Vitaletheine V₄ at concentrations of 1 fg/ml and 1 pg/ml stabilized between 70 and 80% of the cells in culture for an entire month, at which time the cells were frozen for forthcoming functional studies. No cells remained in control cultures, i.e., those lacking vitaletheine V₄, by day 6 of the study. Unlike the control cells whose ability to exclude trypan blue dropped precipitously from the first day in culture, 97±3% of the cells in the vitaletheine V₄-supplemented media were viable after 30 days in culture, i.e., they excluded the dye.

### Example VII

### Treatment of Murine Cloudman S-91 Melanoma with Vitalethine

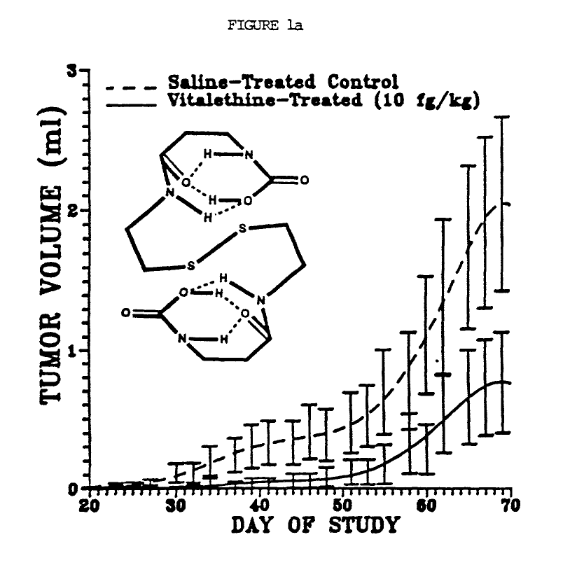

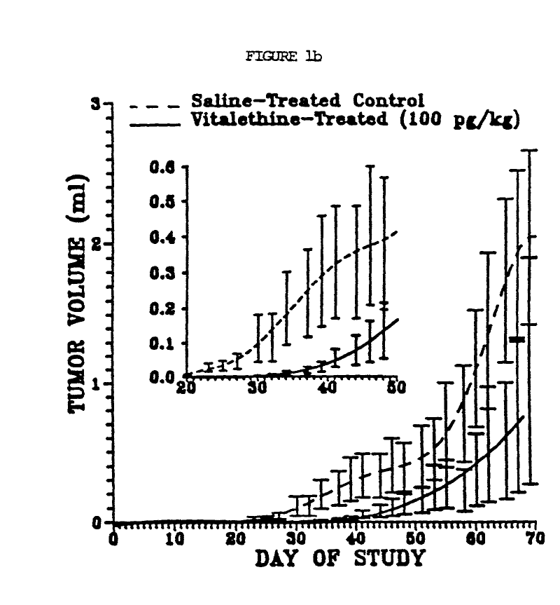

When injected intraperitonealy three times per week (10 femtogram/kg mouse), beginning on the second day after tumor inoculation, vitalethine significantly diminishes tumor volume (˜70%) compared to controls receiving inoculations of tumor cells and injections of saline only (a). This regimen is predicated on the observation that certain enzymes thought to be responsive to these compounds, and which may pay a role in the reported results, are induced 48 hours after chemical stimulation. Vitalethine diminishes average tumor volume substantially over two ranges of concentrations as illustrated by responses to 10 fg/kg mouse (FIG. 1a) and at 100 pg/kg mouse (FIG. 1b). Data presented is calculated from measured tumor diameters, including mice with no tumor. Error bars indicate standard error of the mean.

Cloudman S-91 murine melanoma cells (American Type Culture Collection #53.1, Clone M-3) from a (C X DBA)F1 male mouse were grown in 75 ml flasks (Corning Glass Works, Corning, N.Y., USA) containing Ham's F12 medium supplemented with 15% fetal bovine serum, penicillin (100 U/ml), and streptomycin (100 μg/ml), all commercially available from Sigma Chemical Company, St. Louis, Mo., USA. Cultures were incubated at 37° C. under 5.5% carbon dioxide initially at 6×10⁶ cells per ml for two days, with a medium change after one day. Cultures were then trypsinized, split into two fresh flasks at one half the cell density, above, and maintained for one week prior to injection in female (DBA X BALB c) mice (CD2F1/Hsd from Harlan Sprague Dawley, Inc., Indianapolis, Ind., USA). For inoculation, cells were first trypsinized, washed 3 times in phosphate buffered saline, and diluted to 1×10⁵ cells/100 μl phosphate buffered saline prior to subcutaneous injection in the flank.

The compounds were dissolved in water, filtered through an appropriate sterilizing filter (0.22 μm non-pyrogenic, μ Star LB™ from Costar®, Cambridge, Mass., USA), diluted to the desired concentration in sterile, physiological saline (0.1 ml), and injected intraperitoneally with a 27 gauge, ⅜ inch allergy syringe (Becton Dickinson, Rutherford, N.J., USA); by gently lifting the skin on the abdomen of supine mice and injecting horizontally, puncture of peritoneal organs is avoided, thereby minimizing trauma to the mice.

### Example VIII

### Treatment of Murine NS-1 Myeloma with Vitalethine Modulator

FIG. 2a: Weights of tumor-inoculated, compound-treated mice are significantly lower when treated with certain concentrations of the benzyl derivative compared to tumor-inoculated controls injected with saline (carrier) only, and approximate those of saline-injected mice not challenged with tumor (not shown). FIG. 2b: The weight differences between drug-treated, tumor-inoculated mice and their corresponding drug-treated controls not challenged with tumor is dependent upon the concentration of the benzyl derivative. Instead of the direct measurement of the tumor diameter as in the melanoma model (FIG. 1), myeloma development is estimated by an increase in the weights of mice (reflecting ascites and solid tumor formation) relative to saline- and compound-injected controls. Groups are normalized to the average weight of each group at the start of the study, and bars are standard error of the mean.

NS-1 mylemoa cells (ATCC TIB 18, P3/NS1/1-AG4-1) were employed as inoculant in the BALBc/J mice model; these cells are about 90% effective in establishing myelomas in mice according to the exemplified procedure, and the untreated myelomas are substantially fatal within about two weeks. The cells were grown for several passages (preferably one week) in a sterile environment consisting of RPMI 1640 (Whittaker M.A. Bioproducts, Walkersville, Md., USA) containing 10% fetal calf serum (Hyclone Laboratories, Logan, Utah, USA), 2 mM L-glutamine, 5,000 units of penicillin, and 5 mg streptomycin in 75 cm² polystyrene tissue-culture flasks (Corning Glassworks, Corning, N.Y., USA), in a humidified chamber at 37° C. and under 6% CO₂. To assure NS-1 propagation in vivo, it is essential to remove DMSO (they cryostatic agent dimethyl sulfoxide) through several medium changes and dilutions; this also serves to maintain the cells in log-phase growth. Female BALBc/J mice were injected i.p. with 10⁴ cells in 0.1 ml of standard phosphate-buffered saline as soon as possible after weaning, transport, and indexing, as it has been found that the NS-1 cell line employed does not generally perform optimally in animals which are mature or which have equilibrated with their environment. The mice were maintained with Wayne Rodent Blox (Wayne Research Animal Diets, Chicago, Ill., USA) ad. lib. and tap water.

Concentrations of the benzyl derivative of vitaletheine based upon the average body weight of each group of mice were injected i.p. in 0.1 ml sterile physiological saline starting the second day after tumor inoculation, and continuing every Monday, Wednesday, and Friday throughout the study.

### Example IX

### Modulation of Erythropoiesis with Vitalethine

FIG. 3a: Colony formation from human BFU-E initially deprived of erythropoietin (open square) are increased by vitalethine to levels (broken lines) initially containing erythropoietin, but lacking vitalethine (solid square). Colony formation from the early murine progenitors not exposed to vitalethine, and either initially exposed to or initially deprived of erythropoietin are represented by upper and lower triangles, respectively. Vitalethine, depending upon concentration, either enhances or minimizes erythropoiesis from the CFU-E progenitors (solid line). FIG. 3b: Preformed vitaletheine V₄ stimulates colony formation synergistically with erythropoietin at much lower concentrations (from about 10 fg vitaletheine V₄/ml) than the higher concentrations of vitalethine (from about 10 pg/ml) necessary for a similar response (FIG. 3a). Bars are standard error of the mean.

Human bone marrow cells were obtained as surplus from experiments performed on material aspirated from normal volunteers with IRB approval and informed written consent. Peripheral blood cells were obtained from commercially purchased buffy coats or surgical waste (umbilical cord blood). Mouse bone marrow was flushed from femurs and obtained as surplus from experiments performed on C57B1/6 mice with animal committee approval. Human light density cells were separated by centrifugation over Ficoll-daitrizoate (SG 1.075) and depleted of adherent cells by incubation on serum coated plastic. Mouse cells were used without further fractionation. Cells were suspended at a concentration of 1 to 3 million cells per ml of Iscove's medium (IMDM) supplemented with 10% heat-inactivated fetal calf serum (FCS) with varying concentrations of vitalethine or vitaletheine V₄. One unit per ml of erythropoietin and medium without added factors served as positive and negative controls. Initial incubations were carried out for 18 hours at 37° . Cell suspensions were then pelleted and washed, and the cells were resuspended in culture medium for plasma clot cultures similar to that previously described (Dessypris, E. N., Clark, D. A., McKee, L. C., et. al. in N. Engl. J. Med. 309: 690, 1983.) except that fibrinogen was omitted, fetal calf serum replaced human (AB) serum, and human (AB) plasma replaced bovine plasma. The erythropoietin concentration for cultures of CFU-E was one unit per ml and for BFU-E was 3 units per ml. Cultures were continued for the following periods: mouse and human CFU-E for two and seven days, respectively; and mouse and human BFU-E cultures for seven and fourteen days, respectively. Cultures were fixed, harvested, and stained for hemoglobin with benzidine, and scored as previously described.

What is claimed is:

1. A method for treating a mammalian, including human, disease or disorder comprising exposing the cells involved in the disease or disorder to a therapeutic amount of at least one vitaletheine modulator of the formula: 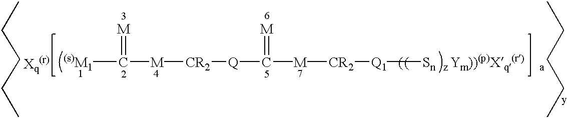
wherein:
the set of double parentheses brackets the portion of the molecule bearing a charge p when z is 1;
M₁—(C═M)—M— (wherein C is the #2 carbon atom) is M₁—(C═M)—M—, M₁═(C—MA)—M—, or M₁—(C—MA)═N—; and —(C═M)—M— (wherein C is the #5 carbon atom) is —(C═M)—M— or —(C—MA)═N—; wherein A is X, −1, or a direct bond, or when —(C═M)—M— is —(C—MA)═N— or the compound is polymeric or internal cyclic or spirocyclic, A is optionally R; and M and M₁ are as defined below;
each R is independently H or a hydrocarbon radical;
X is a biologically-compatible cation or cationic complex;
X′ is a biologically-compatible ion or ionic complex;
M is S, O, N, or NH;
M₁ is S or O with the proviso that M₁ is also optionally N or NH when the modulator is polymeric, or internal cyclic or spirocyclic;
Q is CR₂ or a direct bond;
Q₁ is CR₂, CR₂CR₂, or a direct bond;
Y is O, —[(C═O ]-R, or a direct bond;
a is the absolute value of |r/(r′+p+Σ)| with the proviso that when (r′+p+Σs) is ≧0, at least one q or q′ is zero, such that the sum of any charges on the remainder of the complex is balanced by charges on ion or ions, X or X′, or ions, X and X′;
m is 0 or a whole integer from +1 to +5;
n is 1 or 2 when z is 1, and n is 1 or 1.5 when z is 2;
p is +1, 0, or −1;
q and q′ are each independently +1 or zero;
r and r′ are each independently a whole integer from +1 to +4 or r′ is a whole integer from −1 to −4;
s is −1 or 0;
y is 1 to 40;
z is +1 or +2; and
wherein the modulator has a molecular weight of no more than about 10,000 daltons.

2. The method of claim 1, wherein the treatment further includes exposure of the cells to at least one inhibitor of the metabolism of the modulator or modulators being administered; or at least one agent for enhancing cell propagation; or both.

3. The method of claim 1, wherein the cells are exposed to the modulator by removing the involved cells from the mammal, culturing them in the presence of the modulator for a sufficient amount of time to produce the desired biological effect, and then returning them to the mammal.

4. The method of claim 1, wherein the cells are exposed to the modulator, in vivo.

5. The method of claim 1, wherein the modulator is of the formula: 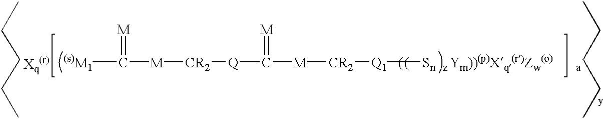
wherein Z_{w}⁽⁰⁾ is a neutral moiety associated with the modulator of claim 1, and w is a whole integer from +1 to +5.

6. The method of claim 1, wherein the modulator is of the formula:

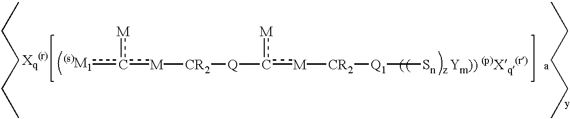

7. The method of claim 5, wherein the modulator is of the formula:

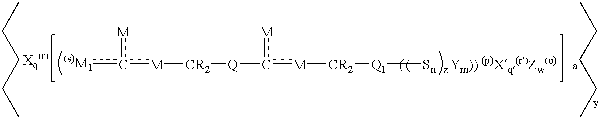

8. The method of claim 1, wherein y in the formula of the modulator is from 1 to about 20.

9. The method of claim 1, wherein the molecular weight of the modulator is no more than about 5,000 daltons.

10. The method of claim 7, wherein the molecular weight of the modulator is no more than about 5,000 daltons.

11. The method of claim 1, wherein the molecular weight of the modulator is at least about 130 daltons.

12. The method of claim 5, wherein the modulator is of the formula:

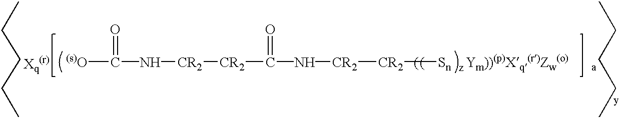

13. The method of claim 12, wherein X in the formula of the modulator is hydronium, H⁺, or Zn⁺².

14. The method of claim 13, wherein R in the formula of the modulator is H.

15. The method of claim 12, wherein the modulator is of the formula: 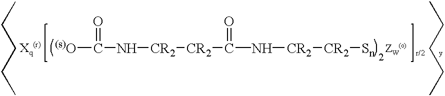
wherein n is 1 or 1.5.

16. The method of claim 12, wherein the modulator is of the formula: 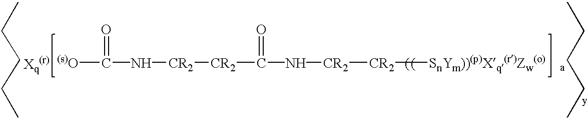
wherein n is 1 or 2, and Y is 0.

17. The method of claim 12, wherein s in the formula of the modulator is −1.

18. The method of claim 15, wherein s in the formula of the modulator is −1.

19. The method of claim 16, wherein s in the formula of the modulator is −1.

20. The method of claim 16, wherein ((SₙYₘ))^{(p)} in the formula of the modulator is an ionized residue of a sulfoxy or thiosulfoxy acid.

21. The method of claim 20, wherein the sulfoxy or thiosulfoxy acid moiety of the modulator is sulfenic, sulfinic, thiosulfenic, thiosulfoxylic, thiosulfurous, or thiosulfuric acid.

22. The method of claim 16, wherein ((SₙYₘ))^{(p)}X′_{q′}^{(r′)}Z_{w}⁽⁰⁾ in the formula of the modulator is SOX′_{q′}, SX′_{q′}, SI, SI₃, S₂O₃X′, SH, or SOH.

23. The method of claim 16, wherein ((SₙYₘ)) in the formula of the modulator is a thioester residue.

24. The method of claim 5, wherein the modulator is of the formula: 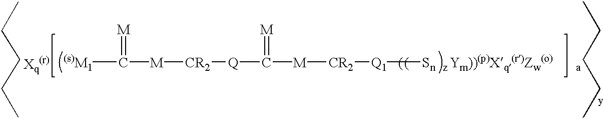
wherein at least one of the expressions, M₁—(C═M)—M— or —(C═M)—M—, is M₁—(C—MA)═N— or M₁═(C—MA)—M—, or —(C—MA)═N—, respectively.

25. The method of claim 24, wherein both of the expressions in the formula of the modulator, M₁—(C═M)—M— and —(C═M)—M—, are M₁—(C—MA)═N— or M₁═(C—MA)—M—, and —(C—MA)═N—, respectively.

26. The method of claim 24, wherein A in the formula of the modulator is X.

27. The method of claim 24, wherein A in the formula of the modulator is R.

28. The method of claim 7, wherein the modulator is of the formula: 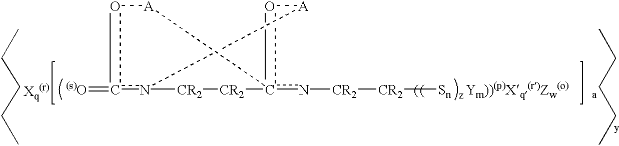
wherein A is associated with the oxygen in the imine or the nitrogen in the amide and the dotted lines depict bond resonances or tautomerisms.

29. The method of claim 28, wherein z in the formula of the modulator is 1.

30. The method of claim 25, wherein z in the formula of the modulator is 2.

31. The method of claim 30, wherein the modulator is of the formula:

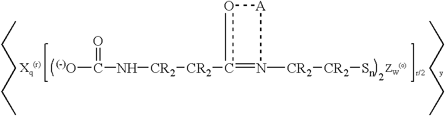

32. The method of claim 28, wherein s in the formula of the modulator is −1.

33. A method for treating a mammalian, including human, disease or disorder comprising exposing the cells involved in the disease or disorder to a therapeutic amount of vitalethine or a physiologically-compatible salt or tautomer thereof.

34. A method for treating a mammalian, including human, disease or disorder comprising exposing the cells involved in the disease or disorder to a therapeutic amount of vitaletheine or a physiologically-compatible salt or tautomer thereof.

35. The method of claim 5, wherein the modulator is an internal cyclic or spirocyclic form of a modulator of claim 5, of the following formula, produced by nucleophilic attack of at least one of the potentially nucleophilic atoms (1,3,6); or at least one of the nucleophilic atoms S, or Y; or at least one of each in a monomer according to claim 5 on at least one of its own doubly-bonded carbon atoms (2,5); according to the following formula:

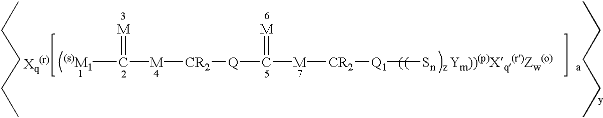

36. The method of claim 35, wherein the modulator is of the formula: 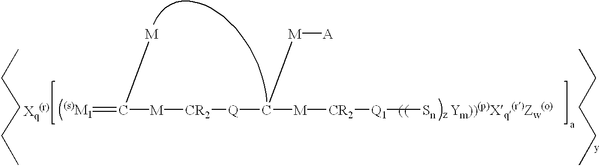
wherein M₁═C—M is M₁═C—M—, and q and s are zero; or M₁═C—M— is M₁—C═N— and s is −1.

37. The method of claim 36, wherein the modulator is of the formula:

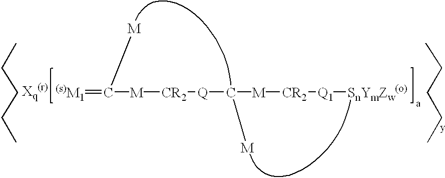

38. The method of claim 36, wherein the modulator is of the formula:

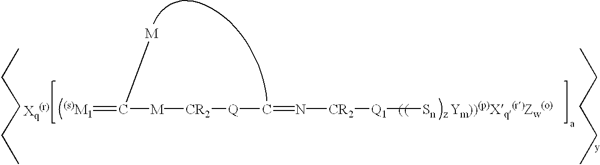

39. The method of claim 35, wherein the modulator is of the formula:

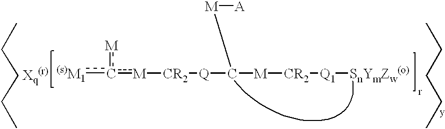

40. The method of claim 35, wherein the modulator is of the formula:

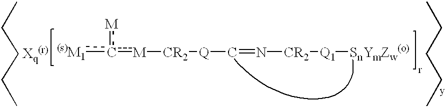

41. The method of claim 35, wherein the modulator is of the formula:

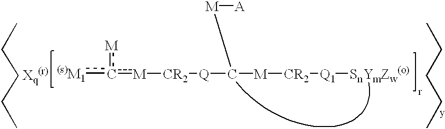

42. The method of claim 35, wherein the modulator is of the formula:

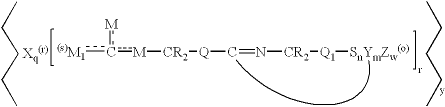

43. The method of claim 35, wherein the modulator is of the formula:

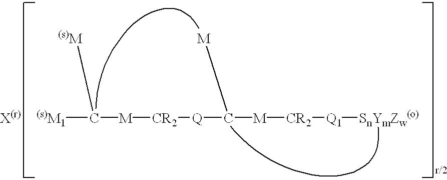

44. The method of claim 35, wherein the modulator is of the formula:

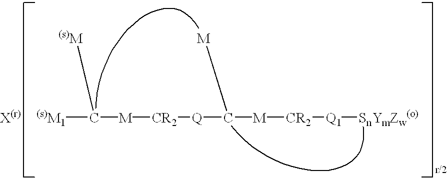

45. The method of claim 35, wherein the modulator is of the formula: 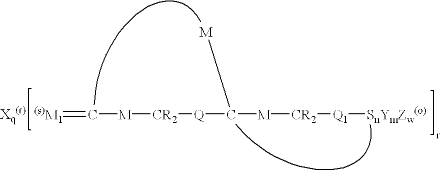
wherein M₁═C—M— is M₁═C—M—, and q and s are zero; or M₁═C—M— is M₁—C═N—, and s is −1.

46. The method of claim 35, wherein the modulator is of the formula: 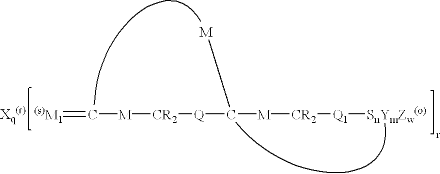
wherein M₁═C—M— is M₁═C—M—, and q and s are zero;, or M₁═C—M— is M₁—C═N—, and s is −1.

47. The method of claim 7, wherein the modulator is an internal cyclic or spirocyclic form of a modulator according to claim 7 of the following formula, produced by nucleophilic attack of at least one of the potentially nucleophilic M atoms (1,3,6); at least one of the nucleophilic atoms S or Y; or at least one of each; in a monomer according to claim 7 on at least one of its own doubly-bonded carbon atoms (2,5) or S, or both:

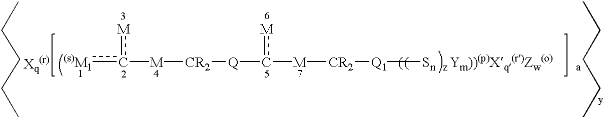

48. The method of claim 47, wherein the modulator is an internal cyclic or spirocyclic form of a modulator according to claim 47 wherein M and M1 are O, produced by nucleophilic attack of at least one nucleophile O, S, or Y, of a monomer according to claim 47 on at least one of its own carbonyl carbon atoms, or S, or both.

49. The method of claim 48, wherein the modulator is of the formula: 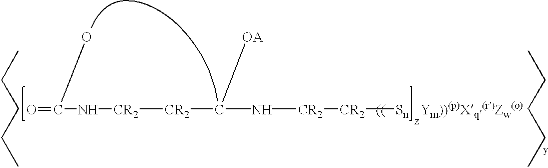
or an imidocarbonate tautomer thereof of the formula:

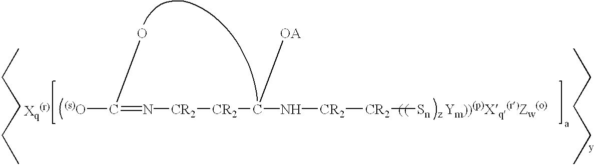

50. The method of claim 48, wherein the modulator is of the formula: 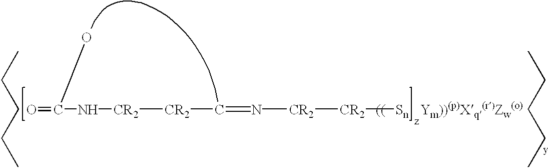
or an imidocarbonate tautomer thereof of the formula:

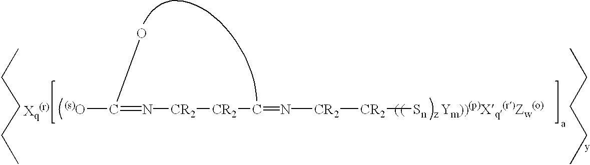

51. The method of claim 48, wherein the modulator is of the formula: 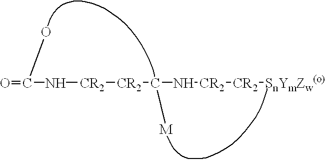
or an imidocarbonate tautomer thereof of the formula:

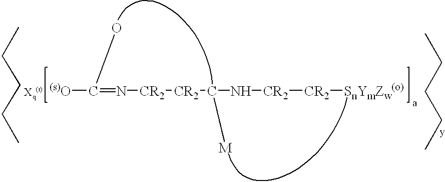

52. The method of claim 48, wherein the modulator is of the formula:

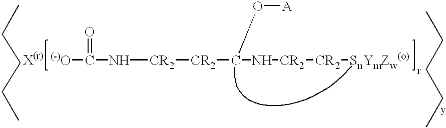

53. The method of claim 48, wherein the modulator is of the formula:

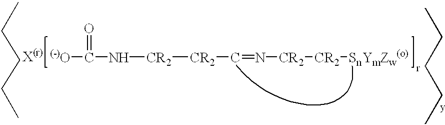

54. The method of claim 48, wherein the modulator is of the formula:

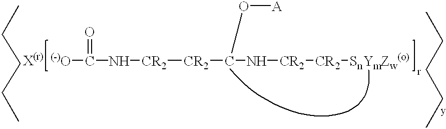

55. The method of claim 48, wherein the modulator is of the formula:

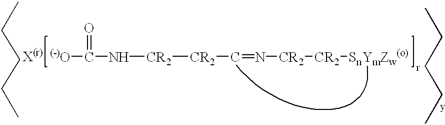

56. The method of claim 48, wherein the modulator is of the formula:

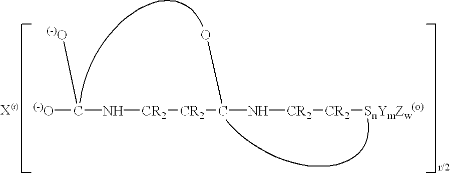

57. The method of claim 48, wherein the modulator is of the formula:

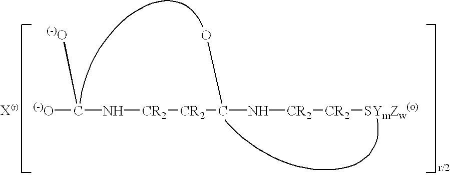

58. The method of claim 48, wherein the modulator is of the formula: 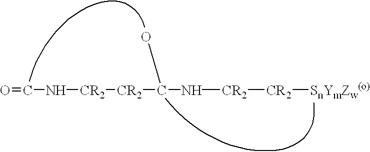
or an imidocarbonate tautomer thereof of the formula:

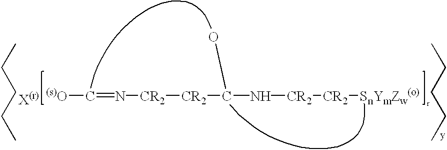

59. The method of claim 48, wherein the modulator is of the formula: 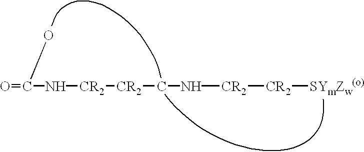
or an imidocarbonate tautomer thereof of the formula:

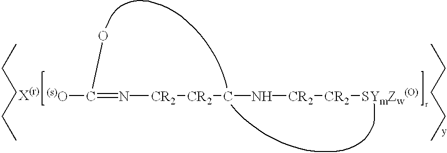

60. The method of claim 1, wherein the modulator is a polymer according to claim 1 wherein y is greater than 1; wherein polymerization is initiated by nucleophilic attack of at least one of the atoms M₁, M (3,6), S, or Y of a first monomer of a modulator of claim 7, on at least one of the doubly-bonded carbon atoms (2,5) of at least one other monomer according to claim 7; and wherein the monomers are of a different formula.

61. The method of claim 5, wherein the modulator is a polymer according to claim 5 wherein y is greater than 1; wherein polymerization is initiated by nucleophilic attack of at least one of the atoms M₁, M (3,6), S, or Y of a first monomer of a modulator of claim 7, on at least one of the doubly-bonded carbon atoms (2,5) of at least one other monomer according to claim 7; wherein the monomers are of the same formula.

62. The method of claim 12, wherein the modulator is a polymer according to claim 12 wherein y is greater than 1; wherein polymerization is initiated by nucleophilic attack of at least one of the atoms O, S, or Y of a first monomer of a modulator according to claim 12, on at least one of the doubly-bonded carbon atoms (2,5) of at least one other monomer according to claim 12 wherein y is 1.

63. A method for treating a mammalian, including human, disease or disorder comprising exposing the cells involved in the disease or disorder to a therapeutic amount of a vitaletheine polymer of the formula: 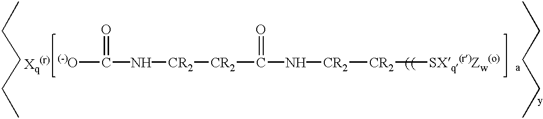
wherein y is from about 2 to 40; R, X, X′, q′, r′, Z, and r are as defined in claim 1; and wherein ⁽⁻⁾O—(C═O)—NH— is ⁽⁻⁾O—(C═O)—NH— and —(C═O)—NH— is —(C—O—)—NH—; or ⁽⁻⁾O—(C═O)—NH— is ⁽⁻⁾O—(C—O—)—NH— and —(C═O)—NH— is —(C═O)—NH—; or both ⁽⁻⁾O—(C═O)—NH— and —(C═O)—NH— are ⁽⁻⁾O—(C—O—)—NH— and —(C—O—)— NH, respectively.

64. The method of claim 60, wherein y in the formula for the vitaletheine polymer is from about 2 to 10.

65. The method of claim 60, wherein y in the formula for the vitaletheine polymer is from about 2 to 4.

66. A method for treating a mammalian, including human, disease or disorder comprising exposing the cells involved in the disease or disorder to a therapeutic amount of an internal cyclic or spirocyclic form of a modulator of the following formula produced by nucleophilic attack of at least one of the potentially nucleophilic atoms (1,3,6) or a nucleophilic atom S or Y of a monomer according to claim 1 on at least one of its own doubly-bonded carbon atoms (2,5), or S, or both:

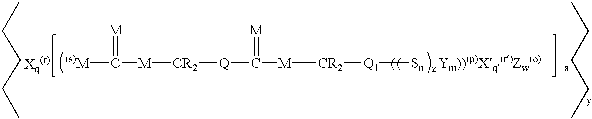

67. A method for treating a mammalian, including human, disease or disorder comprising exposing the cells involved in the disease or disorder to a therapeutic amount of modulator in a cell culture medium including an effective amount of at least one of the modulators of claims 1, 5, 7, 12, 22, 23, 33, 34, 35, 36, 47, 48, 60, 61, 65, or 66; or optionally further exposing the cells to an inhibitor, including β-alethine, of the metabolism of modulator or modulators present in the medium; or optionally further exposing the cells to an agent for enhancing cell propagation; or optionally further exposing the cells to both, an inhibitor of the metabolism and an agent for enhancing cell propagation.

68. A method for treating a mammalian, including human, disease or disorder arising from either inadequate or excessive cell production, comprising exposing the cells involved in the disease or disorder to a therapeutic amount of at least one vitaletheine modulator according to claims 1, 5, 7, 12, 22, 23, 33, 34, 35, 36, 47, 48, 60, 61, 65, or 66; or optionally further exposing the cells to an inhibitor, including β-alethine, of the metabolism of the modulator or modulators present in the medium; or optionally further exposing the cells to an agent for enhancing cell propagation; or optionally further exposing the cells to both, an inhibitor of the metabolism and an agent for enhancing cell propagation.

69. A method for treating a mammalian, including human, disease or disorder arising from either inadequate or excessive cell function, comprising exposing the cells involved in the disease or disorder to a therapeutic amount of at least one vitaletheine modulator according to claims 1, 5, 7, 12, 22, 23, 33, 34, 35, 36, 47, 48, 60, 61, 65, or 66; or optionally further exposing the cells to an inhibitor, including β-alethine, of the metabolism of the modulator or modulators present in the medium; or optionally further exposing the cells to an agent for enhancing cell propagation; or optionally further exposing the cells to both, an inhibitor of the metabolism and an agent for enhancing cell propagation.

70. A method for treating diseases in mammals, including humans, arising from either impaired or aberrant immunological screening, comprising exposing the cells involved in the disease or disorder to a therapeutic amount of at least one vitaletheine modulator according to claims 1, 5, 7, 12, 22, 23, 33, 34, 35, 36, 47, 48, 60, 61, 65, or 66; or optionally further exposing the cells to an inhibitor, including β-alethine, of the metabolism of the modulator or modulators present in the medium; or optionally further exposing the cells to an agent for enhancing cell propagation; or optionally further exposing the cells to both, an inhibitor of the metabolism and an agent for enhancing cell propagation.

71. The method of claim 2, wherein the inhibitor is β-alethine.

72. The method of claim 1, wherein the disease or disorder is acquired immune deficiency syndrome (AIDS), and wherein the treatment comprises administration of the modulator or modulators; or optionally in combination with the administration an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

73. The method of claim 1, wherein the disease or disorder is hypogammaglobulinemia, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

74. The method of claim 1, wherein the disease or disorder is lupus erythematosus, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

75. The method of claim 1, wherein the disease or disorder is rheumatoid arthritis, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

76. The method of claim 1, wherein the disease or disorder is parasite-induced, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

77. The method of claim 1, wherein the disease or disorder is pathogen-(including bacterially, virally, or fungally) induced, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

78. The method of claim 1, wherein the disease or disorder is hormonal, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

79. The method of claim 1, wherein the disease or disorder is thyroid-related, and wherein the treatment comprises administration of the modulator or modulators; or optionally in combination with the administration of an inhibitor, including β-alethine, of the metabolism of the modulator or modulators being administered; or optionally an agent for enhancing cell propagation; or optionally in combination with both, an inhibitor of metabolism and an agent for enhancing cell propagation.

80. The method of claim 1, wherein the disease or disorder is diabetes, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

81. The method of claim 1, wherein the disease or disorder is atherosclerosis or related heart disease, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

82. The method of claim 1, wherein the disease or disorder is cystinosis, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of the metabolism and an agent for enhancing cell propagation.

83. The method of claim 1, wherein the disease or disorder is aging, including progeria, and wherein the treatment comprises administration of the modulator or modulators; or optionally in combination with the administration of an inhibitor, including β-alethine, of the metabolism of the modulator or modulators being administered; or optionally in combination with an agent for enhancing cell propagation; or optionally in combination with both, an inhibitor of metabolism and an agent for enhancing cell propagation.
